(19) United States
(12) Patent Application Publication
(10) Pub. No.: US 2006/0135441 A1
(43) Pub. Date: Jun. 22, 2006

(54) Combinatorial chemotherapy treatment using Na+/K+ ATPase inhibitors

(75) Inventors: Mehran Khodadoust, Brookline, MA (US); Ajay Sharma, Sudbury, MA (US)

Correspondence Address: FISH & NEAVE IP GROUP;ROPES & GRAY LLP, ONE INTERNATIONAL PLACE, BOSTON, MA, 02110-2624, US

(73) Assignee: Bionaut Pharmaceuticals, Inc., Cambridge, MA (US)

(21) Appl. No.: 11/219,636

(22) Filed: Sep. 2, 2005

Related U.S. Application Data
(60) Provisional application No. 60/606,685, filed on Sep. 2, 2004.

(51) Int. Cl. A61K 31/704

(52) U.S. Cl. 514/26

(57) ABSTRACT

The reagent, pharmaceutical formulation, kit, and methods of the invention provides a new approach to alleviate or eliminate certain negative effects associated with the use of certain cancer treatment agents (e.g. chemotherapy therapeutics, etc.) or regimens (e.g. radio therapies, etc.), including stimulation of the hypoxic stress response in tumor cells.

### REFERENCE TO RELATED APPLICATION

This application claims the benefit of the filing date of U.S. Provisional Application Ser. No. 60/606,685, entitled “COMBINATORIAL CHEMOTHERAPY TREATMENTS USING CARDIAC GLYCOSIDES AND OTHER Na/K-ATPASE INHIBITORS,” and filed on Sep. 2, 2004. The teachings of the referenced application are incorporated herein by reference.

### BACKGROUND OF THE INVENTION

HIF-1 is a transcription factor and is critical to survival in hypoxic conditions, both in cancer and cardiac cells. HIF-1 is composed of the O₂— and growth factor-regulated subunit HIF-1α, and the constitutively expressed HIF-1β subunit (arylhydrocarbon receptor nuclear translocator, ARNT), both of which belong to the basic helix-loop-helix (bHLH)-PAS (PER, ARNT, SIM) protein family. So far in the human genome 3 isoforms of the subunit of the transcription factor HIF have been identified: HIF-1, HIF-2 (also referred to as EPAS-1, MOP2, HLF, and HRF), and HIF-3 (of which HIF-32 also referred to as IPAS, inhibitory PAS domain).

Under normoxic conditions, HIF-1α is targeted to ubiquitinylation by pVHL and is rapidly degraded by the proteasome. This is triggered through posttranslational HIF-hydroxylation on specific proline residues (proline 402 and 564 in human HIF-1α protein) within the oxygen dependent degradation domain (ODDD), by specific HIF-prolyl hydroxylases (HPH1-3 also referred to as PHD1-3) in the presence of iron, oxygen, and 2-oxoglutarate. The hydroxylated protein is then recognized by pVHL, which functions as an E3 ubiquitin ligase. The interaction between HIF-1α and pVHL is further accelerated by acetylation of lysine residue 532 through an N-acetyltransferase (ARD1). Concurrently, hydroxylation of the asparagine residue 803 within the C-TAD also occurs by an asparaginyl hydroxylase (also referred to as FIH-1), which by its turn does not allow the coactivator p300/CBP to bind to HIF-1α subunit. In hypoxia HIF-1α remains not hydroxylated and stays away from interaction with pVHL and CBP/p300 (FIG. 6). Following hypoxic stabilization HIF-1α translocates to the nucleus where it heterodimerizes with HIF-1β. The resulting activated HIF-1 drives the transcription of over 60 genes important for adaptation and survival under hypoxia including glycolytic enzymes, glucose transporters Glut-1 and Glut-3, endothelin-1 (ET-1), VEGF (vascular endothelial growth factor), tyrosine hydroxylase, transferrin, and erythropoietin (Brahimi-Horn et al., 2001 Trends Cell Biol 11(11): S32-S36; Beasley et al., 2002 Cancer Res 62(9): 2493-2497; Fukuda et al., 2002 J Biol Chem 277(41): 38205-38211; Maxwell and Ratcliffe, 2002 Semin Cell Dev Biol 13(1): 29-37).

Hypoxia appears to promote tumor growth by promoting cell survival through its induction of angiogenesis and its activation of anaerobic metabolism. The inventors have discovered that certain anti-tumor agents in fact promote an hypoxic stress response in tumor cells, which accordingly should have a direct consequence on clinical and prognostic parameters and create a therapeutic challenge. This hypoxic response includes induction of HIF-1 dependent transcription. The effect of HIF-1 on tumor growth is complex and involves the activation of several adaptive pathways.

It is an object of the present invention to improve the use of those an anti-cancer agent that induces an hypoxic stress response in tumor cells.

### SUMMARY OF THE INVENTION

A salient feature of the present invention is the discovery that certain anti-tumor agents induce an hypoxic stress response in tumor cells, and that Na⁺/K⁺-ATPase inhibitors, such as cardiac glycosides, can be used to reduce that response and improve the efficacy of those anti-tumor agents.

One aspect of the invention provides a pharmaceutical formulation comprising a Na⁺/K⁺-ATPase inhibitor, such as a cardiac glycoside, and an anti-cancer agent that induces an hypoxic stress response in tumor cells, formulated in a pharmaceutically acceptable excipient and suitable for use in humans to treat a neoplastic disorder.

Another aspect of the invention provides a kit for treating a patient having a neoplastic disorder, comprising a Na⁺/K⁺-ATPase inhibitor and an anti-cancer agent that induces an hypoxic stress response in tumor cells, each formulated in premeasured doses for conjoint administration to a patient.

Yet another aspect of the invention provides a method for treating a patient having a neoplastic disorder comprising administering to the patient an effective amount of a Na⁺/K⁺-ATPase inhibitor and an anti-cancer agent that induces an hypoxic stress response in tumor cells.

Still another aspect of the invention provides a method for promoting treatment of patients having a neoplastic disorder, comprising packaging, labeling and/or marketing a Na⁺/K⁺-ATPase inhibitor to be used in conjoint therapy for treating a patient having a neoplastic disorder with an anti-cancer agent that induces an hypoxic stress response in tumor cells.

Another aspect of the invention relates to a method for promoting treatment of patients having a neoplastic disorder, comprising packaging, labeling and/or marketing an anti-cancer agent that induces an hypoxic stress response in tumor cells to be used in conjoint therapy with a Na⁺/K⁺-ATPase inhibitor for treating a patient having a neoplastic disorder.

In certain preferred embodiments, the Na⁺/K⁺-ATPase inhibitor is a cardiac glycoside.

In certain embodiments, the cardiac glycoside, in combination with the anti-cancer agent, has an IC₅₀ for killing one or more different cancer cell lines that is at least 2 fold less relative to the IC₅₀ of the cardiac glycoside alone, and even more preferably at least 5, 10, 50 or even 100 fold less.

In certain embodiments, the cardiac glycoside, in combination with the anti-cancer agent, has an EC₅₀ for treating the neoplastic disorder that is at least 2 fold less relative to the EC₅₀ of the cardiac glycoside alone, and even more preferably at least 5, 10, 50 or even 100 fold less.

In certain embodiments, the cardiac glycoside has an IC₅₀ for killing one or more different cancer cell lines of 500 nM or less, and even more preferably 200 nM, 100 nM, 10 nM or even 1 nM or less.

In certain embodiments, the cardiac glycoside comprises a steroid core with either a pyrone substituent at C17 (the “bufadienolides form”) or a butyrolactone substituent at C17 (the “cardenolide” form).

In certain embodiments, the cardiac glycoside is represented by the general formula:

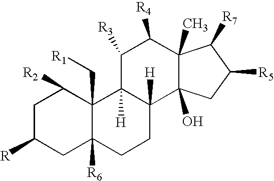

wherein

R represents a glycoside of 1 to 6 sugar residues;

R₁ represents hydrogen, —OH or ═O;

R₂, R₃, R₄, R₅, and R₆ each independently represents hydrogen or —OH;

R₇ represents

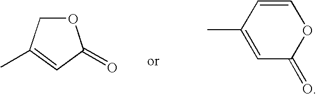

In certain preferred embodiments, the sugar residues are selected from L-rhamnose, D-glucose, D-digitoxose, D-digitalose, D-digginose, D-sarmentose, L-vallarose, and D-fructose. In certain embodiments, these sugars are in the β-conformation. The sugar residues may be acetylated, e.g., to effect the lipophilic character and the kinetics of the entire glycoside. In certain preferred embodiments, the glycoside is 1-4 sugar residues in length.

In certain embodiments, the cardiac glycoside is selected from digitoxigenin, digoxin, lanatoside C, Strophantin K, uzarigenin, desacetyllanatoside A, actyl digitoxin, desacetyllanatoside C, strophanthoside, scillaren A, proscillaridin A, digitoxose, gitoxin, strophanthidiol, oleandrin, acovenoside A, strophanthidine digilanobioside, strophanthidin-d-cymaroside, digitoxigenin-L-rhamnoside, digitoxigenin theretoside, strophanthidin, digoxigenin 3,12-diacetate, gitoxigenin, gitoxigenin 3-acetate, gitoxigenin 3,16-diacetate, 16-acetyl gitoxigenin, acetyl strophanthidin, ouabagenin, 3-epigoxigenin, neriifolin, acetylneriifolin cerberin, theventin, somalin, odoroside, honghelin, desacetyl digilanide, calotropin, calotoxin, convallatoxin, oleandrigenin, bufalin, periplocymarin, digoxin (CP 4072), strophanthidin oxime, strophanthidin semicarbazone, strophanthidinic acid lactone acetate, emicymarin, sannentoside D, sarverogenin, sarmentoside A, sarmentogenin, or a pharmaceutically acceptable salt, ester, amide, or prodrug thereof.

In certain preferred embodiments, the cardiac glycoside is ouabain or proscillaridin.

Other Na⁺/K⁺-ATPase inhibitors are available in the literature. See, for example, U.S. Pat. No. 5,240,714 which describes a non-digoxin-like Na⁺/K⁺-ATPase inhibitory factor. Recent evidence suggests the existence of several endogenous Na⁺/K⁺-ATPase inhibitors in mammals and animals. For instance, marinobufagenin (3,5-dihydroxy-14,15-epoxy bufodienolide) may be useful in the current combinatorial therapies.

Those skilled in the art can also rely on screening assays to identify compounds that have Na⁺/K⁺-ATPase inhibitory activity. PCT Publications WO00/44931 and WO02/42842, for example, teach high-throughput screening assays for modulators of Na⁺/K⁺-ATPases.

The Na⁺/K⁺-ATPase consists of at least two dissimilar subunits, the large α subunit with all known catalytic functions and the smaller glycosylated β subunit with chaperonic function. In addition there may be a small regulatory, so-called FXYD-peptide. Four α peptide isoforms are known and isoform-specific differences in ATP, Na+ and K+ affinities and in Ca2+ sensitivity have been described. Thus changes in The Na⁺/K⁺-ATPase isoform distribution in different tissues, as a function of age and development, electrolytes, hormonal conditions etc. may have important physiological implications. Cardiac glycosides like ouabain are specific inhibitors of the Na⁺/K⁺-ATPase. The four α peptide isoforms have similar high ouabain affinities with Kd of around 1 nM or less in almost all mammalian species. In certain embodiments, the Na+, K+-ATPase inhibitor is more selective for complexes expressed in non-cardiac tissue, relative to cardiac tissue.

In certain embodiments, the anti-cancer agent induces redox-sensitive transcription.

In certain embodiments, the anti-cancer agent induces HIF-1α-dependent transcription.

In certain embodiments, the anti-cancer agent induces expression of one or more of cyclin G2, IGF2, IGF-BP1, IGF-BP2, IGF-BP3, EGF, WAF-1, TGF-α, TGF-β3, ADM, EPO, IGF2, EG-VEGF, VEGF, NOS2, LEP, LRP1, HK1, HK2, AMF/GP1, ENO1, GLUT1, GAPDH, LDHA, PFKBF3, PKFL, MIC1, NIP3, NIX and/or RTP801.

In certain embodiments, the anti-cancer agent induces mitochondrial dysfunction and/or caspase activation.

In certain embodiments, the anti-cancer agent induces cell cycle arrest at G2/M in the absence of said cardiac glycoside.

In certain embodiments, the anti-cancer agent is an inhibitor of chromatin function.

In certain embodiments, the anti-cancer agent is a DNA topoisomerase inhibitor, such as selected from adriamycin, amsacrine, camptothecin, daunorubicin, dactinomycin, doxorubicin, eniposide, epirubicin, etoposide, idarubicin, irinotecan (CPT-11) and mitoxantrone.

In certain embodiments, the anti-cancer agent is a microtubule inhibiting drug, such as a taxane, including paclitaxel, docetaxel, vincristin, vinblastin, nocodazole, epothilones and navelbine.

In certain embodiments, the anti-cancer agent is a DNA damaging agent, such as actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16).

In certain embodiments, the anti-cancer agent is an antimetabolite, such as a folate antagonists, or a nucleoside analog. Exemplary nucleoside analogs include pyrimidine analogs, such as 5-fluorouracil; cytosine arabinoside, and azacitidine. In other embodiments, the nucleoside analog is a purine analog, such as 6-mercaptopurine; azathioprine; 5-iodo-2′-deoxyuridine; 6-thioguanine; 2-deoxycoformycin, cladribine, cytarabine, fludarabine, mercaptopurine, thioguanine, and pentostatin. In certain embodiments, the nucleoside analog is selected from AZT (zidovudine); ACV; valacylovir; famiciclovir; acyclovir; cidofovir; penciclovir; ganciclovir; Ribavirin; ddC; ddI (zalcitabine); lamuvidine; Abacavir; Adefovir; Didanosine; d4T (stavudine); 3TC; BW 1592; PMEA/bis-POM PMEA; ddT, HPMPC, HPMPG, HPMPA, PMEA, PMEG, dOTC; DAPD; Ara-AC, pentostatin; dihydro-5-azacytidine; tiazofurin; sangivamycin; Ara-A (vidarabine); 6-MMPR; 5-FUDR (floxuridine); cytarabine (Ara-C; cytosine arabinoside); 5-azacytidine (azacitidine); HBG [9-(4-hydroxybutyl)guanine], (1S,4R)-4-[2-amino-6-cyclopropyl-amino)-9H-purin-9-yl]-2-cyclopentene-1-methanol succinate (“159U89”), uridine; thymidine; idoxuridine; 3-deazauridine; cyclocytidine; dihydro-5-azacytidine; triciribine, ribavirin, and fludrabine.

In certain embodiments, the nucleoside analog is a phosphate ester selected from the group consisting of: Acyclovir; 1-β-D-arabinofuranosyl-E-5-(2-bromovinyl)uracil; 2′fluorocarbocyclic-2′-deoxyguanosine; 6′-fluorocarbocyclic-2′-deoxyguanosine; 1-(β-D-arabinofuranosyl)-5(E)-(2-iodovinyl)uracil; {(1r-1α,2β,3α)-2-amino-9-(2,3-bis(hydroxymethyl)cyclobutyl)-6H-purin-6-one}Lobucavir; 9H-purin-2-amine, 9-((2-(1-methylethoxy)-1-((1-methylethoxy)methyl)ethoxy)methyl)-(9Cl); trifluorothymidine; 9->(1,3-dihydroxy-2-propoxy)methylguanine (ganciclovir); 5-ethyl-2′-deoxyuridine; E-5-(2-bromovinyl)-2′-deoxyuridine; 5-(2-chloroethyl)-2′-deoxyuridine; buciclovir; 6-deoxyacyclovir; 9-(4-hydroxy-3-hydroxymethylbut-1-yl)guanine; E-5-(2-iodovinyl)-2′-deoxyuridine; 5-vinyl-1-β-D-arabinofuranosyluracil; 1-β-D-arabinofuranosylthymine; 2′-nor-2′deoxyguanosine; and 1-β-D-arabinofuranosyladenine.

In certain embodiments, the nucleoside analog modulates intracellular CTP and/or dCTP metabolism.

In certain preferred embodiments, the nucleoside analog is gemcitabine.

In certain embodiments, the anti-cancer agent is a DNA synthesis inhibitor, such as a thymidilate synthase inhibitors (such as 5-fluorouracil), a dihydrofolate reductase inhibitor (such as methoxtrexate), or a DNA polymerase inhibitor (such as fludarabine).

In certain embodiments, the anti-cancer agent is a DNA binding agent, such as an intercalating agent.

In certain embodiments, the anti-cancer agent is a DNA repair inhibitor.

In certain embodiments, the anti-cancer agent is part of a combinatorial therapy selected from ABV, ABVD, AC (Breast), AC (Sarcoma), AC (Neuroblastoma), ACE, ACe, AD, AP, ARAC-DNR, B-CAVe, BCVPP, BEACOPP, BEP, BIP, BOMP, CA, CABO, CAF, CAL-G, CAMP, CAP, CaT, CAV, CAVE ADD, CA-VP16, CC, CDDP/VP-16, CEF, CEPP(B), CEV, CF, CHAP, ChlVPP, CHOP, CHOP-BLEO, CISCA, CLD-BOMP, CMF, CMFP, CMFVP, CMV, CNF, CNOP, COB, CODE, COMLA, COMP, Cooper Regimen, COP, COPE, COPP, CP-Chronic Lymphocytic Leukemia, CP-Ovarian Cancer, CT, CVD, CVI, CVP, CVPP, CYVADIC, DA, DAT, DAV, DCT, DHAP, DI, DTIC/Tamoxifen, DVP, EAP, EC, EFP, ELF, EMA 86, EP, EVA, FAC, FAM, FAMTX, FAP, F-CL, FEC, FED, FL, FZ, HDMTX, Hexa-CAF, ICE-T, IDMTX/6-MP, IE, IfoVP, IPA, M-2, MAC-III, MACC, MACOP-B, MAID, m-BACOD, MBC, MC, MF, MICE, MINE, mini-BEAM, MOBP, MOP, MOPP, MOPP/ABV, MP-multiple myeloma, MP-prostate cancer, MTX/6-MO, MTX/6-MP/VP, MTX-CDDPAdr, MV-breast cancer, MV-acute myelocytic leukemia, M-VAC Methotrexate, MVP Mitomycin, MVPP, NFL, NOVP, OPA, OPPA, PAC, PAC-I, PA-CL, PC, PCV, PE, PFL, POC, ProMACE, ProMACE/cytaBOM, PRoMACE/MOPP, Pt/VM, PVA, PVB, PVDA, SMF, TAD, TCF, TIP, TTT, Topo/CTX, VAB-6, VAC, VACAdr, VAD, VATH, VBAP, VBCMP, VC, VCAP, VD, VelP, VIP, VM, VMCP, VP, V-TAD, 5+2, 7+3, “8 in 1”.

In certain embodiments, the anti-cancer agent is selected from altretamine, aminoglutethimide, amsacrine, anastrozole, asparaginase, bcg, bicalutamide, bleomycin, buserelin, busulfan, calcium folinate, campothecin, capecitabine, carboplatin, carmustine, chlorambucil, cisplatin, cladribine, clodronate, colchicine, crisantaspase, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

In certain embodiments, the anti-cancer agent is selected from tamoxifen, 4-(3-chloro-4-fluorophenylamino)-7-methoxy-6-(3-(4-α-morpholinyl)propoxy)quinazoline, 4-(3-ethynylphenylamino)-6,7-bis(2-methoxyethoxy)quinazoline, hormones, steroids, steroid synthetic analogs, 17a-ethinylestradiol, diethylstilbestrol, testosterone, prednisone, fluoxymesterone, dromostanolone propionate, testolactone, megestrolacetate, methylprednisolone, methyl-testosterone, prednisolone, triamcinolone, chlorotrianisene, hydroxyprogesterone, aminoglutethimide, estramustine, medroxyprogesteroneacetate, leuprolide, flutamide, toremifene, Zoladex, antiangiogenics, matrix metalloproteinase inhibitors, VEGF inhibitors, ZD6474, SU6668, SU11248, anti-Her-2 antibodies (ZD1839 and OS1774), EGFR inhibitors, EKB-569, Imclone antibody C225, src inhibitors, bicalutamide, epidermal growth factor inhibitors, Her-2 inhibitors, MEK-1 kinase inhibitors, MAPK kinase inhibitors, P13 inhibitors, PDGF inhibitors, combretastatins, MET kinase inhibitors, MAP kinase inhibitors, inhibitors of non-receptor and receptor tyrosine kinases (imatinib), inhibitors of integrin signaling, and inhibitors of insulin-like growth factor receptors.

In certain embodiments, the subject combinations are used to inhibit growth of a tumor cell selected from a pancreatic tumor cell, lung tumor cell, a prostate tumor cell, a breast tumor cell, a colon tumor cell, a liver tumor cell, a brain tumor cell, a kidney tumor cell, a skin tumor cell, an ovarian tumor cell and a leukemic blood cell.

In certain embodiments, the subject combination is used in the treatment of a proliferative disorder selected from renal cell cancer, Kaposi's sarcoma, chronic lymphocytic leukemia, lymphoma, mesothelioma, breast cancer, sarcoma, ovarian carcinoma, rectal cancer, throat cancer, melanoma, colon cancer, bladder cancer, mastocytoma, lung cancer, liver cancer, mammary adenocarcinoma, pharyngeal squamous cell carcinoma, prostate cancer, pancreatic cancer, gastrointestinal cancer, and stomach cancer.

It is contemplated that all embodiments of the invention may be combined with any other embodiment(s) of the invention.

### BRIEF DESCRIPTION OF THE DRAWINGS

FIG. 1. Schematic diagram of using Sentinel Line promoter-less trap vectors to generate active genetic sites expressing drug selection markers and/or reporters.

FIG. 2. Schematic diagram of creating a Sentinel Line by sequential isolation of cells resistant to positive and negative selection drugs

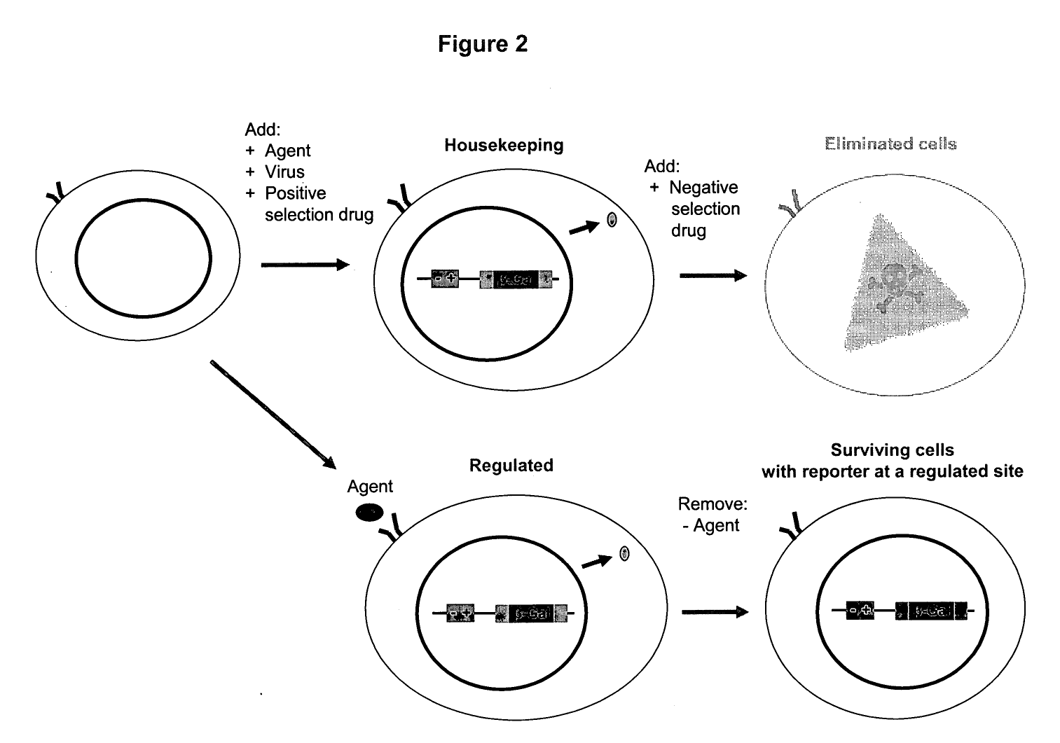

FIG. 3. Adaptation of a cancer cell to hypoxia, which leads to activation of multiple survival factors. The HIF family acts as a master switch transcriptionally activating many genes and enabling factors necessary for glycolytic energy metabolism, angiogenesis, cell survival and proliferation, and erythropoiesis. The level of HIF proteins present in the cell is regulated by the rate of their synthesis in response to factors such as hypoxia, growth factors, androgens and others. Degradation of HIF depends in part on levels of reactive oxygen species (ROS) in the cell. ROS leads to ubiquitylation and degradation of HIF.

FIG. 4. FACS Analysis of Sentinel Lines. Sentinel Lines were developed by transfecting A549 (NSCLC lung cancer) and Panc-1 (pancreatic cancer) cell lines with gene-trap vectors containing E. coli LacZ-encoded β-galactosidase (β-gal) as the reporter gene. The 1-gal activity in Sentinel Lines (green) was measured by flow cytometry using a fluorogenic substrate fluoresescein di-beta-D-galactopyranoside (FDG). The autofluorescence of untransfected control cells is shown in purple. The graphs indicate frequency of cells (y-axis) and intensity of fluorescence (x-axis) in log scale. The bar charts on the right depict median fluorescent units of the FACS curves. They indicate a high level of reporter activity at the targeted site.

FIG. 5. Western Blot analysis of HIF1α expression indicates that cardiac glycoside compounds inhibit HIF1α expression.

FIG. 6. Demonstrates that BNC1 inhibits HIF1α synthesis.

FIG. 7. Demonstrates that BNC1 induces ROS production and inhibits HIF-1α induction in tumor cells.

FIG. 8. Demonstrates that the cardiac glycoside compounds BNC1 and BNC4 directly or indirectly inhibits in tumor cells the secretion of the angiogenesis factor VEGF.

FIG. 9. These four charts show FACS analysis of response of a NSCLC Sentinel Line (A549), when treated 40 hrs with four indicated agents. Control (untreated) is shown in purple. Arrow pointing to the right indicates increase in reporter activity whereas inhibitory effect is indicated by arrow pointing to the left. The results indicate that standard chemotherapy drugs turn on survival response in tumor cells.

FIG. 10. Effect of BNC4 on Gemcitabine-induced stress responses visualized by A549 Sentinel Lines™.

FIG. 11. Pharmacokinetic analysis of BNC1 delivered by osmotic pumps. Osmotic pumps (Model 2002, Alzet Inc) containing 200 μl of BNC1 at 50, 30 or 20 mg/ml in 50% DMSO were implanted subcutaneously into nude mice. Mice were sacrificed after 24, 48 or 168 hrs, and plasma was extracted and analyzed for BNC1 by LC-MS. The values shown are average of 3 animals per point.

FIG. 12. Shows effect of BNC1 alone or in combination with standard chemotherapy on growth of xenografted human pancreatic tumors in nude mice.

FIG. 13. Shows anti-tumor activity of BNC1 and Cytoxan against Caki-1 human renal cancer xenograft.

FIG. 14. Shows anti-tumor activity of BNC1 alone or in combination with Carboplatin in A549 human non-small-cell-lung carcinoma.

FIG. 15. Titration of BNC1 to determine minimum effective dose effective against Panc-1 human pancreatic xenograft in nude mice. BNC1 (sc, osmotic pumps) was tested at 10, 5 and 2 mg/ml.

FIG. 16. Combination of BNC1 with Gemcitabine is more effective than either drug alone against Panc-1 xenografts.

FIG. 17. Combination of BNC1 with 5-FU is more effective than either drug alone against Panc-1 xenografts.

FIG. 18. Comparison of BNC1 and BNC4 in inhibiting hypoxia-mediated HIF-1α induction in human tumor cells (Hep3B cells).

FIG. 19. Comparison of BNC1 and BNC4 in inhibiting hypoxia-mediated HIF-1α induction in human tumor cells (Caki-1 and Panc-1 cells).

FIG. 20. BNC4 blocks HIF-1α induction by a prolyl-hydroxylase inhibitor under normoxia.

### DETAILED DESCRIPTION OF THE INVENTION

I. Overview

The present invention is based in part on the discovery that certain anti-tumor agents in fact promote an hypoxic stress response in tumor cells. For instance, such anti-cancer agents induce expression of one or more of cyclin G2, IGF2, IGF-BP1, IGF-BP2, IGF-BP3, EGF, WAF-1, TGF-α, TGF-β3, ADM, EPO, IGF2, EG-VEGF, VEGF, NOS2, LEP, LRP1, HK1, HK2, AMF/GP1, ENO1, GLUT1, GAPDH, LDHA, PFKBF3, PKFL, MIC1, NIP3, NIX and/or RTP801. By promoting cell survival through its induction of angiogenesis and its activation of anaerobic metabolism, it is believed that the activation of an hypoxic stress response would be counteractive to the other anti-cancer activities of these drugs. A salient feature of the present invention is the discovery that Na⁺/K⁺-ATPase inhibitors (e.g. cardiac glycosides) can be used to reduce the induced hypoxic stress response and improve the efficacy of those anti-tumor agents.

II. Definitions

As used herein the term “animal” refers to mammals, preferably mammals such as humans. Likewise, a “patient” or “subject” to be treated by the method of the invention can mean either a human or non-human animal.

As used herein, the term “cancer” refers to any neoplastic disorder, including such cellular disorders as, for example, renal cell cancer, Kaposi's sarcoma, chronic leukemia, prostate cancer, breast cancer, sarcoma, pancreatic cancer, ovarian carcinoma, rectal cancer, throat cancer, melanoma, colon cancer, bladder cancer, mastocytoma, lung cancer, mammary adenocarcinoma, myeloma, lymphoma, pharyngeal squamous cell carcinoma, and gastrointestinal or stomach cancer. Preferably, the cancer which is treated in the present invention is melanoma, lung cancer, breast cancer, pancreatic cancer, prostate cancer, colon cancer, or ovarian cancer.

The “growth state” of a cell refers to the rate of proliferation of the cell and the state of differentiation of the cell.

As used herein, “hyperproliferative disease” or “hyperproliferative disorder” refers to any disorder which is caused by or is manifested by unwanted proliferation of cells in a patient. Hyperproliferative disorders include but are not limited to cancer, psoriasis, rheumatoid arthritis, lamellar ichthyosis, epidermolytic hyperkeratosis, restenosis, endometriosis, and abnormal wound healing.

As used herein, “proliferating” and “proliferation” refer to cells undergoing mitosis.

As used herein, “unwanted proliferation” means cell division and growth that is not part of normal cellular turnover, metabolism, growth, or propagation of the whole organism. Unwanted proliferation of cells is seen in tumors and other pathological proliferation of cells, does not serve normal function, and for the most part will continue unbridled at a growth rate exceeding that of cells of a normal tissue in the absence of outside intervention. A pathological state that ensues because of the unwanted proliferation of cells is referred herein as a “hyperproliferative disease” or “hyperproliferative disorder.”

As used herein, “transformed cells” refers to cells that have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control. For purposes of this invention, the terms “transformed phenotype of malignant mammalian cells” and “transformed phenotype” are intended to encompass, but not be limited to, any of the following phenotypic traits associated with cellular transformation of mammalian cells: immortalization, morphological or growth transformation, and tumorigenicity, as detected by prolonged growth in cell culture, growth in semi-solid media, or tumorigenic growth in immuno-incompetent or syngeneic animals.

III. Exemplary Embodiments

A. Exemplary Cardiac Glycosides

The inventors have demonstrated that Na⁺/K⁺-ATPase inhibitors (e.g. cardiac glycosides) are effective in suppressing hypoxia-induced gene expression, such as in cancer cells. For example, Na⁺/K⁺-ATPase inhibitors (e.g. cardiac glycosides) are effective in suppressing EGF, insulin and/or IGF-responsive gene expression in various growth factor responsive cancer cell lines. As another example, the inventors have observed that Na⁺/K⁺-ATPase inhibitors (e.g. cardiac glycosides) are effective in suppressing HIF-responsive gene expression in cancer cell lines and furthermore, Na⁺/K⁺-ATPase inhibitors (e.g. cardiac glycosides) are shown to have potent antiproliferative effects in cancer cell lines.

The term “cardiac glycoside” or “cardiac steroid” is used in the medical field to refer to a category of compounds tending to have positive inotropic effects on the heart. As a general class of compounds, cardiac glycosides comprise a steroid core with either a pyrone or butenolide substituent at C17 (the “pyrone form” and “butenolide form”). Additionally, cardiac glycosides may optionally be glycosylated at C3. Most cardiac glycosides include one to four sugars attached to the 3β-OH group. The sugars most commonly used include L-rhamnose, D-glucose, D-digitoxose, D-digitalose, D-digginose, D-sarmentose, L-vallarose, and D-fructose. In general, the sugars affect the pharmacokinetics of a cardiac glycoside with little other effect on biological activity. For this reason, aglycone forms of cardiac glycosides are available and are intended to be encompassed by the term “cardiac glycoside” as used herein. The pharmacokinetics of a cardiac glycoside may be adjusted by adjusting the hydrophobicity of the molecule, with increasing hydrophobicity tending to result in greater absorbtion and an increased half-life. Sugar moieties may be modified with one or more groups, such as an acetyl group.

A large number of cardiac glycosides are known in the art for the purpose of treating cardiovascular disorders. Given the significant number of cardiac glycosides that have proven to have anticancer effects in the assays disclosed herein, it is expected that most or all of the cardiac glycosides used for the treatment of cardiovascular disorders may also be used for treating proliferative disorders. Examples of preferred cardiac glycosides include ouabain, digitoxigenin, digoxin and lanatoside C. Additional examples of cardiac glycosides include: Strophantin K, uzarigenin, desacetyllanatoside A, actyl digitoxin, desacetyllanatoside C, strophanthoside, scillaren A, proscillaridin A, digitoxose, gitoxin, strophanthidiol, oleandrin, acovenoside A, strophanthidine digilanobioside, strophanthidin-d-cymaroside, digitoxigenin-L-rhamnoside, digitoxigenin theretoside, strophanthidin, digoxigenin 3,12-diacetate, gitoxigenin, gitoxigenin 3-acetate, gitoxigenin 3,16-diacetate, 16-acetyl gitoxigenin, acetyl strophanthidin, ouabagenin, 3-epigoxigenin, neriifolin, acetylneriifolin cerberin, theventin, somalin, odoroside, honghelin, desacetyl digilanide, calotropin and calotoxin. Cardiac glycosides may be evaluated for effectiveness in the treatment of cancer by a variety of methods, including, for example: evaluating the effects of a cardiac glycoside on expression of a HIF-responsive gene in a cancer cell line or evaluating the effects of a cardiac glycoside on cancer cell proliferation.

Notably, cardiac glycosides affect proliferation of cancer cell lines at a concentration well below the known toxicity level. The IC₅₀ measured for ouabain across several different cancer cell lines ranged from about 15 nM to about 600 nM, or 80 nM to about 300 nM. The concentration at which a cardiac glycoside is effective as part of an antiproliferative treatment may be further decreased by combination with an additional agent that negatively regulates HIF-responsive genes, such as a redox effector or a steroid signal modulator. For example, as shown herein, the concentration at which a cardiac glycoside (e.g. ouabain or proscillaridin) is effective for inhibiting proliferation of cancer cells is decreased 5-fold by combination with a steroid signal modulator (Casodex). Therefore, in certain embodiments, the invention provides combination therapies of cardiac glycosides with, for example, steroid signal modulators and/or redox effectors. Additionally, cardiac glycosides may be combined with radiation therapy, taking advantage of the radiosensitizing effect that many cardiac glycosides have.

B. Exemplary Anti-Cancer Agents

Pharmaceutical agents that may be used in the subject combination therapy with Na⁺/K⁺-ATPase inhibitors (e.g. cardiac glycosides) include, merely to illustrate: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These anti-cancer agents may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors, epidermal growth factor (EGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-1) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; chromatin disruptors.

These anti-cancer agents are used by itself with an HIF inhibitor, or in combination. Many combinatorial therapies have been developed in prior art, including but not limited to those listed in Table 1.

TABLE 1
Exemplary conventional combination cancer chemotherapy

| Name | Therapeutic agents |
|---|---|
| ABV | Doxorubicin, Bleomycin, Vinblastine |
| ABVD | Doxorubicin, Bleomycin, Vinblastine, |
| | Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, |
| | Procarbazine, Prednisone |
| BEACOPP | Bleomycin, Etoposide, Doxorubicin, |
| | Cyclophosphamide, Vincristine, Procarbazine, |
| | Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, |
| | Prednisone, Asparaginase |
| CAMP | Cyclophosphamide, Doxorubicin, Methotrexate, |
| | Procarbazine |
| CAP | Cyclophosphamide, Doxorubicin, Cisplatin |
| CaT | Carboplatin, Paclitaxel |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with |
| | or without/Bleomycin |
| CEV | Cyclophosphamide, Etoposide, Vincristine |
| CF | Cisplatin, Fluorouracil or Carboplatin |
| | Fluorouracil |
| CHAP | Cyclophosphamide or Cyclophosphamide, |
| | Altretamine, Doxorubicin, Cisplatin |
| ChlVPP | Chlorambucil, Vinblastine, Procarbazine, |
| | Prednisone |
| CHOP | Cyclophosphamide, Doxorubicin, Vincristine, |
| | Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, |
| | Prednisone |
| CMFVP | Cyclophosphamide, Methotrexate, Fluorouracil, |
| | Vincristine, Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, |
| | Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, |
| | Leucovorin, Cytarabine |
| COMP | Cyclophosphamide, Vincristine, Methotrexate, |
| | Prednisone |
| Cooper Regimen | Cyclophosphamide, Methotrexate, Fluorouracil, |
| | Vincristine, Prednisone |
| COP | Cyclophosphamide, Vincristine, Prednisone |
| COPE | Cyclophosphamide, Vincristine, Cisplatin, |
| | Etoposide |
| COPP | Cyclophosphamide, Vincristine, Procarbazine, |
| | Prednisone |
| CP(Chronic | Chlorambucil, Prednisone |
| lymphocytic leukemia) | |
| CP (Ovarian Cancer) | Cyclophosphamide, Cisplatin |
| CT | Cisplatin, Paclitaxel |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, |
| | Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, |
| | Fluorouracil |
| ICE-T | Ifosfamide, Carboplatin, Etoposide, Paclitaxel, |
| | Mesna |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, |
| | Prednisone, Melphalan |
| MAC-III | Methotrexate, Leucovorin, Dactinomycin, |
| | Cyclophosphamide |
| MACC | Methotrexate, Doxorubicin, Cyclophosphamide, |
| | Lomustine |
| MACOP-B | Methotrexate, Leucovorin, Doxorubicin, |
| | Cyclophosphamide, |
| | Vincristine, Bleomycin, Prednisone |
| MAID | Mesna, Doxorubicin, Ifosfamide, Dacarbazine |
| m-BACOD | Bleomycin, Doxorubicin, Cyclophosphamide, |
| | Vincristine, Dexamethasone, Methotrexate, |
| | Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, |
| | Prednisone |
| MOPP/ABV | Mechlorethamine, Vincristine, Procarbazine, |
| | Prednisone, Doxorubicin, Bleomycin, Vinblastine |
| MP (multiple | Melphalan, Prednisone |
| myeloma) | |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, |
| | Prednisone |
| MTX-CDDPAdr | Methotrexate, Leucovorin, Cisplatin, |
| | Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, |
| | Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PC | Paclitaxel, Carboplatin or Paclitaxel, |
| | Cisplatin |
| PCV | Lomustine, Procarbazine, Vincristine |
| PE | Paclitaxel, Estramustine |
| PFL | Cisplatin, Fluorouracil, Leucovorin |
| POC | Prednisone, Vincristine, Lomustine |
| ProMACE | Prednisone, Methotrexate, Leucovorin, |
| | Doxorubicin, Cyclophosphamide, Etoposide |
| ProMACE/cytaBOM | Prednisone, Doxorubicin, Cyclophosphamide, |
| | Etoposide, Cytarabine, Bleomycin, |
| | Vincristine, Methotrexate, Leucovorin, |
| | Cotrimoxazole |
| PRoMACE/MOPP | Prednisone, Doxorubicin, Cyclophosphamide, |
| | Etoposide, Mechlorethamine, Vincristine, |
| | Procarbazine, Methotrexate, Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, |
| | Asparaginase |
| SMF | Streptozocin, Mitomycin, Fluorouracil |
| TAD | Mechlorethamine, Doxorubicin, Vinblastine, |
| | Vincristine, Bleomycin, Etoposide, Prednisone |
| TCF | Paclitaxel, Cisplatin, Fluorouracil |
| TIP | Paclitaxel, Ifosfamide, Mesna, Cisplatin |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, |
| | Cisplatin, Bleomycin |
| VAC | Vincristine, Dactinomycin, Cyclophosphamide |
| VACAdr | Vincristine, Cyclophosphamide, Doxorubicin, |
| | Dactinomycin, Vincristine |
| VAD | Vincristine, Doxorubicin, Dexamethasone |
| VATH | Vinblastine, Doxorubicin, Thiotepa, |
| | Flouxymesterone |
| VBAP | Vincristine, Carmustine, Doxorubicin, |
| | Prednisone |
| VBCMP | Vincristine, Carmustine, Melphalan, |
| | Cyclophosphamide, Prednisone |
| VC | Vinorelbine, Cisplatin |
| VCAP | Vincristine, Cyclophosphamide, Doxorubicin, |
| | Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, |
| | Prednisone |
| VP | Etoposide, Cisplatin |
| V-TAD | Etoposide, Thioguanine, Daunorubicin, |
| | Cytarabine |
| 5 + 2 | Cytarabine, Daunorubicin, Mitoxantrone |
| 7 + 3 | Cytarabine with/, Daunorubicin or Idarubicin |
| | or Mitoxantrone |
| “8 in 1” | Methylprednisolone, Vincristine, Lomustine, |
| | Procarbazine, Hydroxyurea, Cisplatin, |
| | Cytarabine, Dacarbazine |

In addition to conventional anti-cancer agents, the agent of the subject method can also be compounds and antisense RNA, RNAi or other polynucleotides to inhibit the expression of the cellular components that contribute to unwanted cellular proliferation that are targets of conventional chemotherapy. Such targets are, merely to illustrate, growth factors, growth factor receptors, cell cycle regulatory proteins, transcription factors, or signal transduction kinases.

The method of present invention is advantageous over combination therapies known in the art because it allows conventional anti-cancer agent to exert greater effect at lower dosage. In preferred embodiment of the present invention, the effective dose (ED₅₀) for a anti-cancer agent or combination of conventional anti-cancer agents when used in combination with a Na⁺/K⁺-ATPase inhibitor (e.g. cardiac glycoside) is at least 5 fold less than the ED₅₀ for the anti-cancer agent alone. Conversely, the therapeutic index (TI) for such anti-cancer agent or combination of such anti-cancer agent when used in combination with a Na⁺/K⁺-ATPase inhibitor (e.g. cardiac glycoside) is at least 5 fold greater than the TI for conventional anti-cancer agent regimen alone.

C. Other Treatment Methods

In yet other embodiments, the subject method combines a Na⁺/K⁺-ATPase inhibitor (e.g. cardiac glycoside) with radiation therapies, including ionizing radiation, gamma radiation, or particle beams.

D. Administration

The Na⁺/K⁺-ATPase inhibitor (e.g. cardiac glycoside), or a combination containing a Na⁺/K⁺-ATPase inhibitor (e.g. cardiac glycoside) may be administered orally, parenterally by intravenous injection, transdermally, by pulmonary inhalation, by intravaginal or intrarectal insertion, by subcutaneous implantation, intramuscular injection or by injection directly into an affected tissue, as for example by injection into a tumor site. In some instances the materials may be applied topically at the time surgery is carried out. In another instance the topical administration may be ophthalmic, with direct application of the therapeutic composition to the eye.

In a preferred embodiment, the subject Na⁺/K⁺-ATPase inhibitors (e.g. cardiac glycosides) are administered to a patient by using osmotic pumps, such as Alzet® Model 2002 osmotic pump. Osmotic pumps provides continuous delivery of test agents, thereby eliminating the need for frequent, round-the-clock injections. With sizes small enough even for use in mice or young rats, these implantable pumps have proven invaluable in predictably sustaining compounds at therapeutic levels, avoiding potentially toxic or misleading side effects.

To meet different therapeutic needs, ALZET's osmotic pumps are available in a variety of sizes, pumping rates, and durations. At present, at least ten different pump models are available in three sizes (corresponding to reservoir volumes of 100 μL, 200 μL and 2 mL) with delivery rates between 0.25 μL/hr and 10 μL/hr and durations between one day to four weeks.

While the pumping rate of each commercial model is fixed at manufacture, the dose of agent delivered can be adjusted by varying the concentration of agent with which each pump is filled. Provided that the animal is of sufficient size, multiple pumps may be implanted simultaneously to achieve higher delivery rates than are attainable with a single pump. For more prolonged delivery, pumps may be serially implanted with no ill effects. Alternatively, larger pumps for larger patients, including human and other non-human mammals may be custom manufactured by scaling up the smaller models.

The materials are formulated to suit the desired route of administration. The formulation may comprise suitable excipients include pharmaceutically acceptable buffers, stabilizers, local anesthetics, and the like that are well known in the art. For parenteral administration, an exemplary formulation may be a sterile solution or suspension; For oral dosage, a syrup, tablet or palatable solution; for topical application, a lotion, cream, spray or ointment; for administration by inhalation, a microcrystalline powder or a solution suitable for nebulization; for intravaginal or intrarectal administration, pessaries, suppositories, creams or foams. Preferably, the route of administration is parenteral, more preferably intravenous.

### EXEMPLIFICATION

The following examples are for illustrative purpose only, and should in no way be construed to be limiting in any respect of the claimed invention.

The exemplary cardiac glycosides used in following studies are referred to as BNC1 and BNC4.

BNC1 is ouabain or g-Strophanthin (STRODIVAL®), which has been used for treating myocardial infarction. It is a colorless crystal with predicted IC₅₀ of about 0.009-0.35 μg/mL and max. plasma concentration of about 0.03 μg/mL. According to the literature, its plasma half-life in human is about 20 hours, with a range of between 5-50 hours. Its common formulation is injectable. The typical dose for current indication (i.v.) is about 0.25 mg, up to 0.5 mg/day.

BNC4 is proscillaridin (TALUSIN®), which has been approved for treating chronic cardiac insufficiency in Europe. It is a colorless crystal with predicted IC₅₀ of about 0.002-0.008 μg/mL and max. plasma concentration of about 0.001 μg/mL. According to the literature, its plasma half-life in human is about 40 hours. Its common available formulation is a tablet of 0.25 or 0.5 mg. The typical dose for current indication (p.o.) is about 1.5 mg/day.

### Example I

### Sentinel Line Plasmid Construction and Virus Preparation

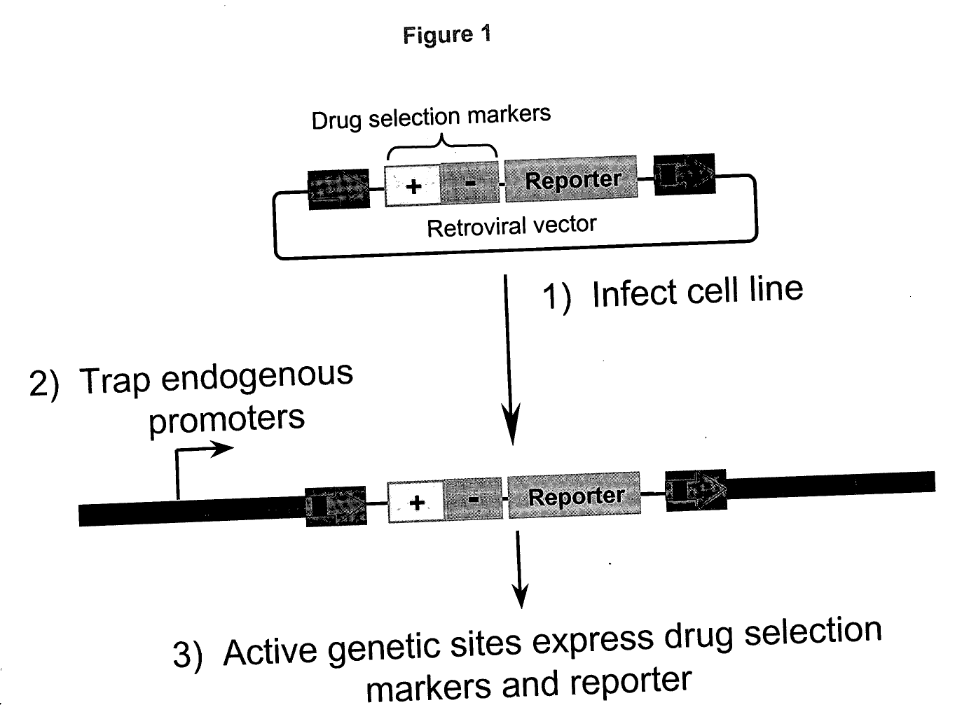

FIG. 1 is a schematic drawing of the Sentinel Line promoter trap system, and its use in identifying regulated genetic sites and in reporting pathway activity. Briefly, the promoter-less selection markers (either positive or negative selection markers, or both) and reporter genes (such as beta-gal) are put in a retroviral vector (or other suitable vectors), which can be used to infect target cells. The randomly inserted retroviral vectors may be so positioned that an active upstream heterologous promoter may initiate the transcription and translation of the selectable markers and reporter gene(s). The expression of such selectable markers and/or reporter genes is indicative of active genetic sites in the particular host cell.

In one exemplary embodiment, the promoter trap vector BV7 was derived from retrovirus vector pQCXIX (BD Biosciences Clontech) by replacing sequence in between packaging signal (Psi⁺) and 3′ LTR with a cassette in an opposite orientation, which contains a splice acceptor sequence derived from mouse engrailed 2 gene (SA/en2), an internal ribosomal entry site (IRES), a LacZ gene, a second IRES, and fusion gene TK:Sh encoding herpes virus thymidine kinase (HSV-tk) and phleomycin followed by a SV40 polyadenylation site. BV7 was constructed by a three-way ligation of three equal molar DNA fragments. Fragment 1 was a 5 kb vector backbone derived from PQCXIX by cutting plasmid DNA extracted from a Dam-bacterial strain with Xho I and Cla I (Dam-bacterial strain was needed here because Cla I is blocked by overlapping Dam methylation). Fragment 2 was a 2.5 kb fragment containing an IRES and a TK:Sh fusion gene derived from plasmid pIREStksh by cutting Dam-plasmid DNA with Cla I and Mlu I. pIREStksh was constructed by cloning TK:Sh fragment from pMODtksh (InvivoGen) into pIRES (BD Biosciences Clontech). Fragment 3 was a 5.8 kb SA/en2-IRES-LacZ fragment derived from plasmid pBSen2IRESLacZ by cutting with BssH II (compatible end to Mlu I) and Xho I. pBSen2IRESLacZ was constructed by cloning IRES fragment from pIRES and LacZ fragment from pMODLacZ (InvivoGen) into plasmid pBSen2.

To prepare virus, packaging cell line 293T was co-transfected with three plasmids BV7, pVSV-G (BD Biosciences Clontech) and pGag-Pol (BD Biosciences Clontech) in equal molar concentrations by using Lipofectamine 2000 (InvitroGen) according to manufacturer's protocol. First virus “soup” (supernatant) was collected 48 hours after transfection, second virus “soup” was collected 24 hours later. Virus particles were pelleted by centrifuging at 25,000 rpm for 2 hours at 4° C. Virus pellets were re-dissolved into DMEM/10% FBS by shaking overnight. Concentrated virus solution was aliquot and used freshly or frozen at −80° C.

### Example II

### Sentinel Line Generation

Target cells were plated in 150 mm tissue culture dishes at a density of about 1×10⁶/plate. The following morning cells were infected with 250 μl of Bionaut Virus #7 (BV7) as prepared in Example I, and after 48 hr incubation, 20 μg/ml of phleomycin was added. 4 days later, media was changed to a reduced serum (2% FBS) DMEM to allow the cells to rest. 48 h later, ganciclovir (GCV) (0.4 μM, sigma) was added for 4 days (media was refreshed on day 2). One more round of phleomycin selection followed (20 μg/ml phleomycin for 3 days). Upon completion, media was changed to 20% FBS DMEM to facilitate the outgrowths of the clones. 10 days later, clones were picked and expanded for further analysis and screening.

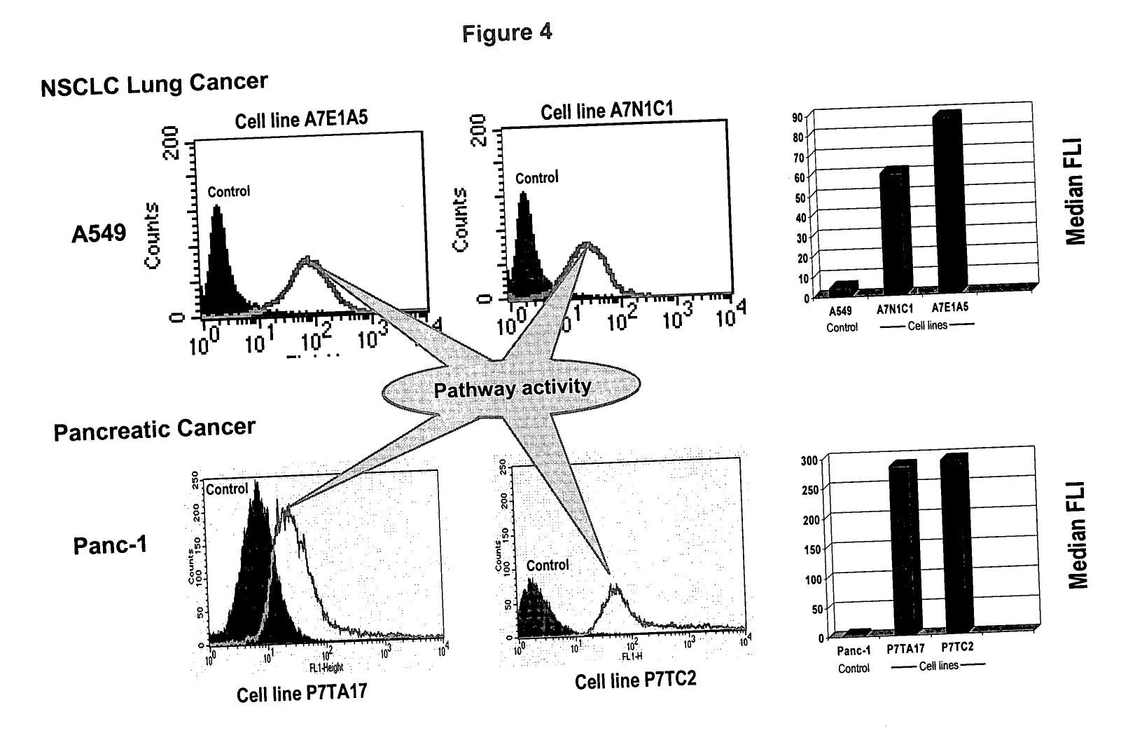

Usig this method, several Sentinel Lines were generated to report activity of genetic sites activated by hypoxia pathways (FIG. 4). These Sentinel lines were generated by transfecting A549 (NSCLC lung cancer) and Panc-1 (pancreatic cancer) cell lines with the subject gene-trap vectors containing E. coli LacZ-encoded β-galactosidase (β-gal) as the reporter gene (FIG. 4). The β-gal activity in Sentinel Lines (green) was measured by flow cytometry using a fluorogenic substrate fluoresescein di-beta-D-galactopyranoside (FDG). The autofluorescence of untransfected control cells is shown in purple. The graphs indicate frequency of cells (y-axis) and intensity of fluorescence (x-axis) in log scale. The bar charts on the right depict median fluorescent units of the FACS curves. They indicate a high level of reporter activity at the targeted site.

### Example III

### Cell Culture and Hypoxic Conditions

All cell lines can be purchased from ATCC, or obtained from other sources.

A549 (CCL-185) and Panc-1 (CRL-1469) were cultured in Dulbecco's Modified Eagle's Medium (DMEM), Caki-1 (HTB-46) in McCoy's 5a modified medium, Hep3B (HB-8064) in MEM-Eagle medium in humidified atmosphere containing 5% CO₂ at 37° C. Media was supplemented with 10% FBS (Hyclone; SH30070.03), 100 μg/ml penicillin and 50 μg/ml streptomycin (Hyclone).

To induce hypoxia conditions, cells were placed in a Billups-Rothenberg modular incubator chamber and flushed with artificial atmosphere gas mixture (5% CO₂, 1% O₂, and balance N₂). The hypoxia chamber was then placed in a 37° C. incubator. L-mimosine (Sigma, M-0253) was used to induce hypoxia-like HIF1-alpha expression. Proteosome inhibitor, MG132 (Calbiochem, 474791), was used to protect the degradation of HIF1-alpha. Cycloheximide (Sigma, 4859) was used to inhibit new protein synthesis of HIF1-alpha. Catalase (Sigma, C3515) was used to inhibit reactive oxygen species (ROS) production.

### Example IV

### Identification of Trapped Genes

Once a Sentinel Line with a desired characteristics was established, it might be helpful to determine the active promoter under which control the markers/reporter genes are expressed. To do so, total RNAs were extracted from cultured Sentinel Line cells by using, for example, RNA-Bee RNA Isolation Reagent (TEL-TEST, Inc.) according to the manufacturer's instructions. Five prime ends of the genes that were disrupted by the trap vector BV7 were amplified by using BD SMART RACE cDNA Amplification Kit (BD Biosciences Clontech) according to the manufacturer's protocol. Briefly, 1 μg total RNA prepared above was reverse-transcribed and extended by using BD PowerScriptase with 5′ CDS primer and BD SMART II Oligo both provided by the kit. PCR amplification were carried out by using BD Advantage 2 Polymerase Mix with Universal Primer A Mix provided by the kit and BV7 specific primer 5′Rsa/ires (gacgcggatcttccgggtaccgagctcc, 28 mer). 5′Rsa/ires located in the junction of SA/en2 and IRES with the first 7 nucleotides matching the last 7 nucleotides of SA/en2 in complementary strand. 5′ RACE products were cloned into the TA cloning vector pCR2.1 (InvitroGen) and sequenced. The sequences of the RACE products were analyzed by using the BLAST program to search for homologous sequences in the database of GenBank. Only those hits which contained the transcript part of SA/en2 were considered as trapped genes.

Using this method, the upstream promoters of several Sentinel Lines generated in Example II were identified (see below). The identity of these trapped genes validate the clinical relevance of these Sentinel Lines™, and can be used as biomarkers and surrogate endpoints in clinical trials.

| Sentinel | | |
|---|---|---|
| Lines | Genetic Sites | Gene Profile |
| A7N1C1 | Essential Antioxidant | Tumor cell-specific |
| | | gene, over expressed |
| | | in lung tumor cells |
| A7N1C6 | Chr. 3, BAC, | novel |
| | map to 3p | |
| A7I1C1 | Pyruvate Kinase | Described biomarker |
| | (PKM 2), Chr. 15 | for NSCLC |
| A6E2A4 | 6q14.2-16.1 | Potent angiogenic |
| | | activity |
| A7I1D1 | Chr. 7, BAC | novel |

### Example V

### Western Blots

For HIF1-alpha Western blots, Hep3B cells were seeded in growth medium at a density of 7×10⁶ cells per 100 mm dish. Following 24-hour incubation, cells were subjected to hypoxic conditions for 4 hours to induce HIF1-alpha expression together with an agent such as 1 μM BNC1. The cells were harvested and lysed using the Mammalian Cell Lysis kit (Sigma, M-0253). The lysates were centrifuged to clear insoluble debris, and total protein contents were analyzed with BCA protein assay kit (Pierce, 23225). Samples were fractionated on 3-8% Tris-Acetate gel (Invitrogen NUPAGE system) by sodium dodecyl sulfate (SDS)-polyacrylamide gel electropherosis and transferred onto nitrocellulose membrane. HIF1-alpha protein was detected with anti-HIF1-alpha monoclonal antibody (BD Transduction Lab, 610959) at a 1:500 dilution with an overnight incubation at 4° C. in Tris-buffered solution-0.1% Tween 20 (TBST) containing 5% dry non-fat milk. Anti-Beta-actin monoclonal antibody (Abcam, ab6276-100) was used at a 1:5000 dilution with a 30-minute incubation at room temperature. Immunoreactive proteins were detected with stabilized goat-anti mouse HRP conjugated antibody (Pierce, 1858413) at a 1:10,000 dilution. The signal was developed using the West Femto substrate (Pierce, 34095).

We examined the inhibitory effect of BNC1 on HIF-1 alpha synthesis. 24 hours prior to treatment, Hep3B cells were seeded in growth medium. To show that BNC1 inhibits HIF 1-alpha expression in a concentration dependent manner, cells were treated with 1 μM BNC1 together with the indicated amount of MG132 under hypoxic conditions for 4 hours. To understand specifically the impact of BNC1 on HIF-1 alpha synthesis, Hep3B cells were treated with MG132 and 1 μM BNC under normoxic conditions for the indicated time points. The observed expression is accounted by protein synthesis.

We examined the role of BNC1 on the degradation rate of HIF-1 alpha. 24 hours prior to treatment, Hep3B cells were seeded in growth medium. The cells were placed in hypoxic conditions for 4 hours for HIF1-alpha accumulation. The protein synthesis inhibitor, cycloheximide (100 μM) together with 1 μM BNC1 were added to the cells and kept in hypoxic conditions for the indicate time points.

To induce HIF1-alpha expression using an iron chelator, L-mimosine was added to Hep3B cells, seeded 24 hours prior, and placed under normoxic conditions for 24 hours.

### Example VI

### Sentinel Line Reporter Assays

The expression level of beta-galactosidase gene in sentinel lines was determined by using a fluorescent substrate fluorescein di-B-D-Galactopyranside (FDG, Marker Gene Tech, #M0250) introduced into cells by hypotonic shock. Cleavage by beta-galactosidase results in the production of free fluorescein, which is unable to cross the plasma membrane and is trapped inside the beta-gal positive cells. Briefly, the cells to be analyzed are trypsinized, and resuspended in PBS containing 2 mM FDG (diluted from a 10 mM stock prepared in 8:1:1 mixture of water: ethanol: DMSO). The cells were then shocked for 4 minutes at 37° C. and transferred to FACS tubes containing cold 1×PBS on ice. Samples were kept on ice for 30 minutes and analyzed by FACS in FL1 channel.

### Example VII

### Testing Standard Chemotherapeutic Agents

Sentinel Line cells with beta-galactosidase reporter gene were plated at 1×10⁵ cells/10 cm dish. After overnight incubation, the cells were treated with standard chemotherapeutic agents, such as mitoxantrone (8 nM), paclitaxel (1.5 nM), carboplatin (15 μM), gemcitabine (2.5 nM), in combination with one or more BNC compounds, such as BNC1 (10 nM), BNC2 (2 μM), BNC3 (100 μM) and BNC4 (10 nM), or a targeted drug, Iressa (4 μM). After 40 hrs, the cells were trypsinized and the expression level of reporter gene was determined by FDG loading.

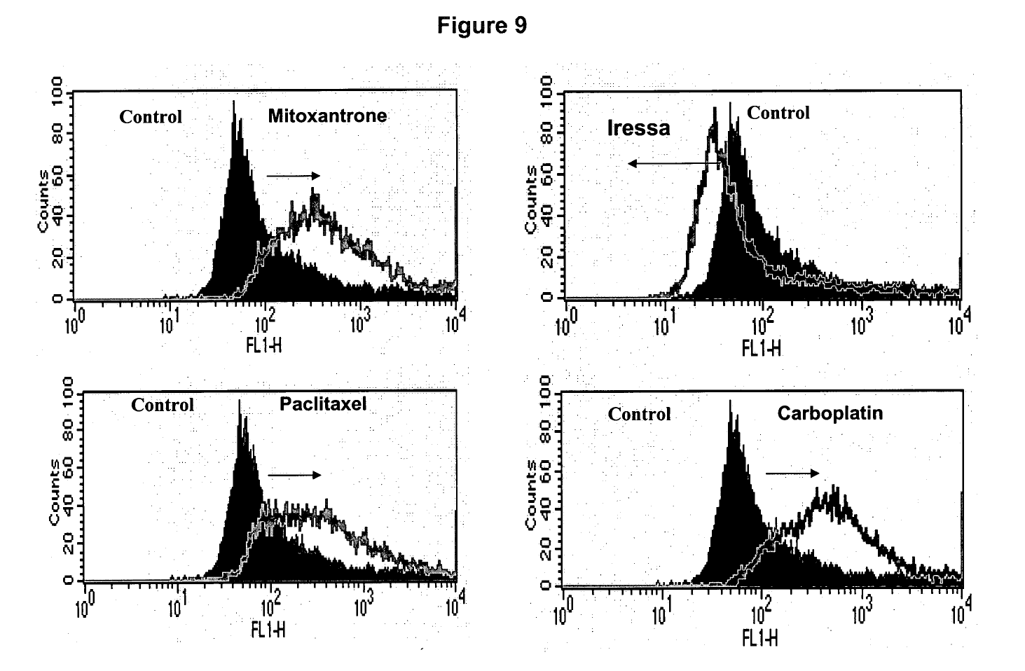

When tested in the Sentinel Lines, mitoxanthrone, paclitaxel, and carboplatin each showed increases in cell death and reporter activity (see FIG. 9). No effect had been expected from the cytotoxic agents because of their nonspecific mechanisms of action (MOA), making their increased reporter activity in HIF-sensitive cell lines surprising. These results provide a previously unexplored link between the development of chemotherapy resistance and induction of the hypoxia response in cells treated with anti-neoplastic agents. Iressa, on the other hand, a known blocker of EGFR-mediated HIF-1 induction, showed a reduction in reporter activity when tested. The Sentinel Lines thus provide a means to differentiate between a cytotoxic agent and a targeted drug.

### Example VIII

### Pharmacokinetic (PK) Analysis

The following protocol can be used to conduct pharmacokinetic analysis of any compounds of the invention. To illustrate, BNC1 is used as an example.

Nude mice were dosed i.p. with 1, 2, or 4 mg/kg of BNC1. Venous blood samples were collected by cardiac puncture at the following 8 time points: 5 min, 15 min, 30 min, 45 min, 1 hr, 2 hr, 4 hr, 8 hr, and 24 hr. For continuous BNC1 treatment, osmotic pumps (such as Alzet® Model 2002) were implanted s.c. between the shoulder blades of each mouse. Blood was collected at 24 hr, 48 hr and 72 hr. Triplicate samples per dose (i.e. three mice per time point per dose) were collected for this experiment.

Approximately 0.100 mL of plasma was collected from each mouse using lithium heparin as anticoagulant. The blood samples were processed for plasma as individual samples (no pooling). The samples were frozen at −70° C. (±10° C.) and transferred on dry ice for analysis by HPLC.

For PK analysis plasma concentrations for each compound at each dose were analyzed by non-compartmental analysis using the software program WinNonlin®. The area under the concentration vs time curve AUC (0-Tf) from time zero to the time of the final quantifiable sample (Tf) was calculated using the linear trapezoid method. AUC is the area under the plasma drug concentration-time curve and is used for the calculation of other PK parameters. The AUC was extrapolated to infinity (0-Inf) by dividing the last measured concentration by the terminal rate constant (k), which was calculated as the slope of the log-linear terminal portion of the plasma concentrations curve using linear regression. The terminal phase half-life (t_{1/2}) was calculated as 0.693/k and systemic clearance (Cl) was calculated as the dose(mg/kg)/AUC(Inf). The volume of distribution at steady-state (Vss) was calculated from the formula:
Vₛₛ=dose(AUMC)/(AUC)²

where AUMC is the area under the first moment curve (concentration multiplied by time versus time plot) and AUC is the area under the concentration versus time curve. The observed maximum plasma concentration (Cₘₐₓ) was obtained by inspection of the concentration curve, and Tₘₐₓ is the time at when the maximum concentration occurred.

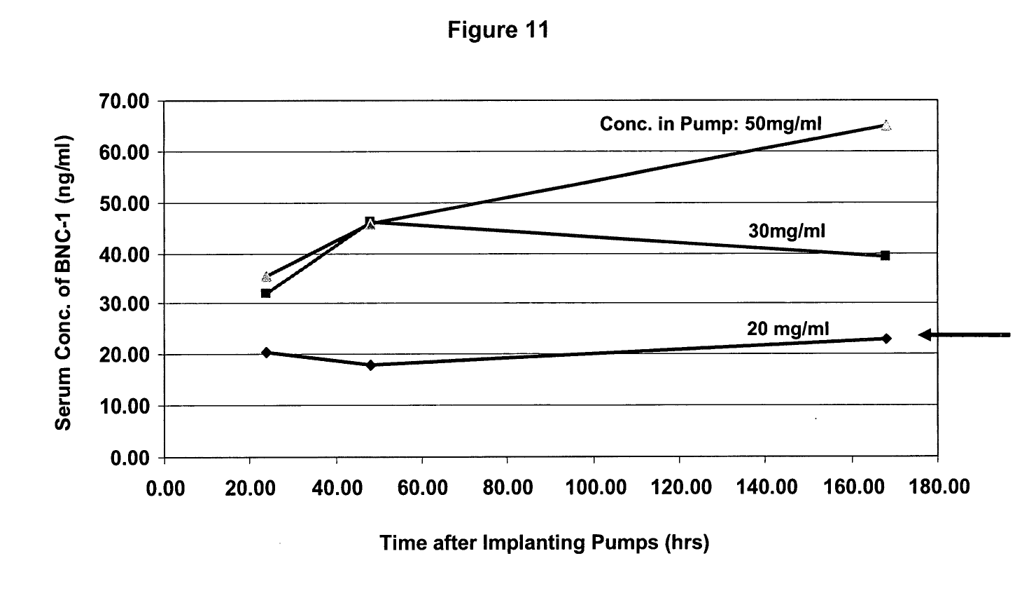

FIG. 11 shows the result of a representative pharmacokinetic analysis of BNC1 delivered by osmotic pumps. Osmotic pumps (Model 2002, Alzet Inc) containing 200 μl of BNC1 at 50, 30 or 20 mg/ml in 50% DMSO were implanted subcutaneously into nude mice. Mice were sacrificed after 24, 48 or 168 hrs, and plasma was extracted and analyzed for BNC1 by LC-MS. The values shown are average of 3 animals per point.

### Example IX

### Human Tumor Xenograft Models

Female nude mice (nu/nu) between 5 and 6 weeks of age weighing approximately 20 g were implanted subcutaneously (s.c.) by trocar with fragments of human tumors harvested from s.c. grown tumors in nude mice hosts. When the tumors were approximately 60-75 mg in size (about 10-15 days following inoculation), the animals were pair-matched into treatment and control groups. Each group contains 8-10 mice, each of which was ear tagged and followed throughout the experiment.

The administration of drugs or controls began the day the animals were pair-matched (Day 1). Pumps (Alzet® Model 2002) with a flow rate of 0.5 μl/hr were implanted s.c. between the shoulder blades of each mice. Mice were weighed and tumor measurements were obtained using calipers twice weekly, starting Day 1. These tumor measurements were converted to mg tumor weight by standard formula, (W²×L)/2. The experiment is terminated when the control group tumor size reached an average of about 1 gram. Upon termination, the mice were weighed, sacrificed and their tumors excised. The tumors were weighed and the mean tumor weight per group was calculated. The change in mean treated tumor weight/the change in mean control tumor weight×100 (dT/dC) is subtracted from 100% to give the tumor growth inhibition (TGI) for each group.

### Example X

### Cardiac Glycoside Compounds Inhibits HIF-1α Expression

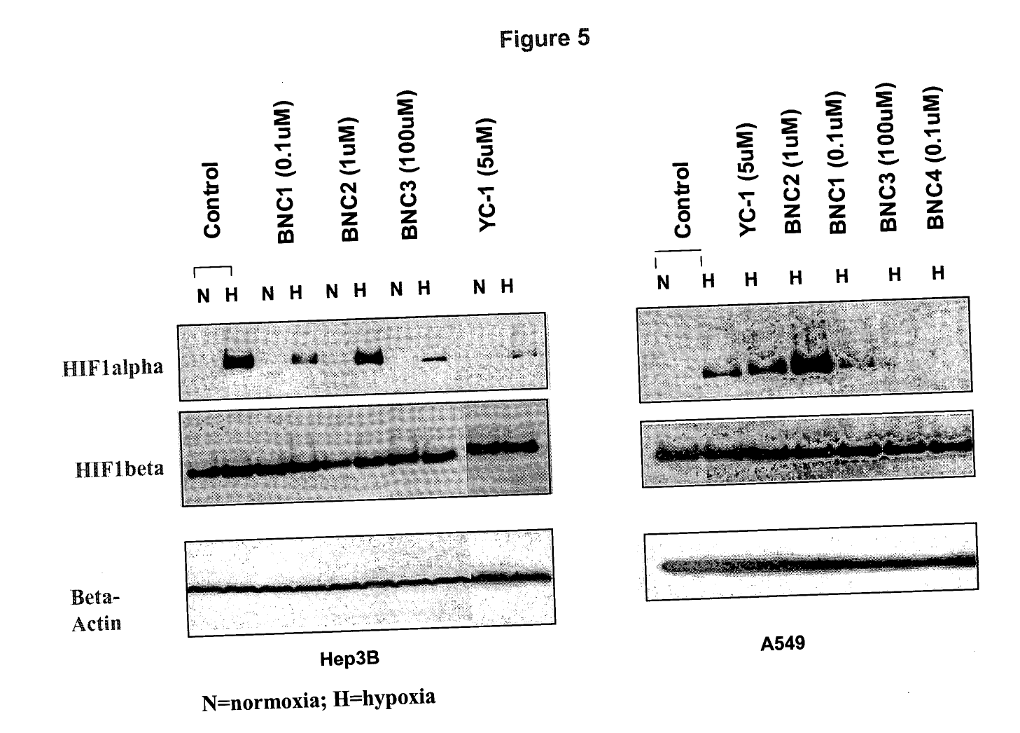

Cardiac glycoside compounds of the invention targets and inhibits the expression of HIF 1α based on Western Blot analysis using antibodies specific for HIF1α (FIG. 5).

Hep3B or A549 cells were cultured in complete growth medium for 24 hours and treated for 4 hrs with the indicated cardiac glycoside compounds or controls under normoxia (N) or hypoxia (H) conditions. The cells were lysed and proteins were resolved by SDS-PAGE and transferred to a nylon membrane. The membrane was immunoblotted with anti-HIF1α and anti-HIF1β MAb, and anti-beta-actin antibodies.

In Hep3B cells, various effective concentrations of BNC compounds (cardiac glycoside compounds of the invention) inhibits the expression of HIF-1α, but not HIF-1β. The basic observation is the same, with the exception of BNC2 at 1 μM concentration.

To study the mechanism of HIF-1α inhibition by the subject cardiac glycoside compounds, Hep3B cells were exposed to normoxia or hypoxia for 4 hrs in the presence or absence of: an antioxidant enzyme and reactive oxygen species (ROS) scavenger catalase (1000 U), prolyl-hydroxylase (PHD) inhibitor L-mimosine, or proteasome inhibitor MG132 as indicated. HIF1α and β-actin protein level was determined by western blotting.

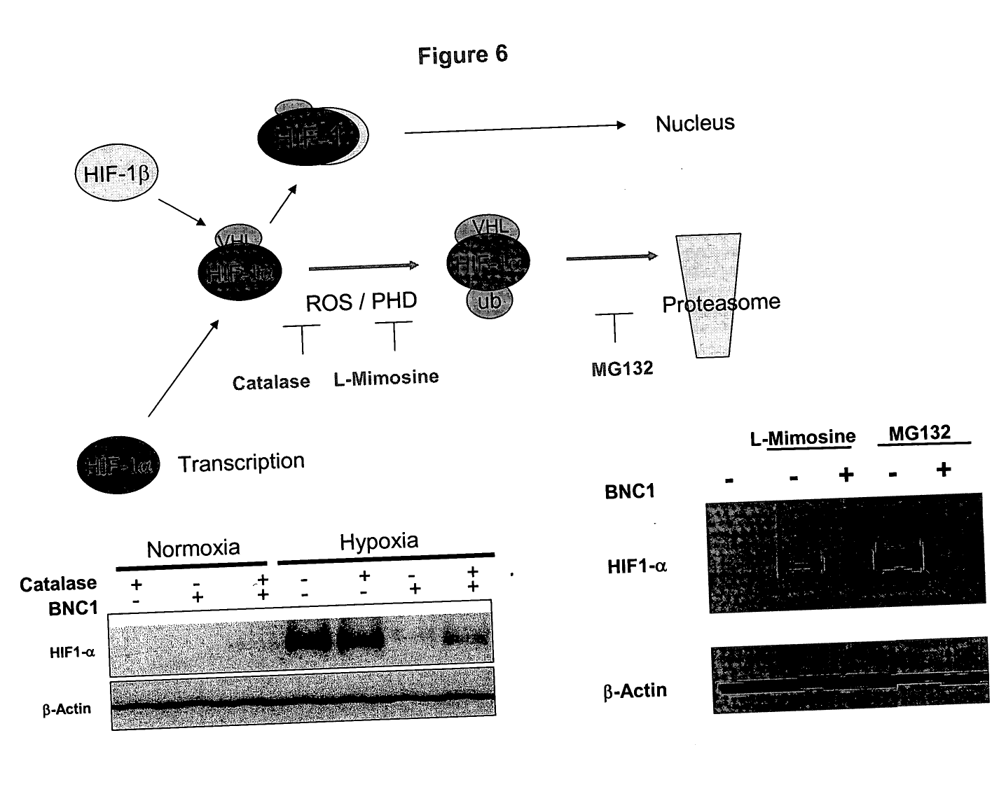

FIG. 6 indicates that the cardiac glycoside compound BNC1 may inhibits steady state HIF-1α level through inhibiting the synthesis of HIF-1α.

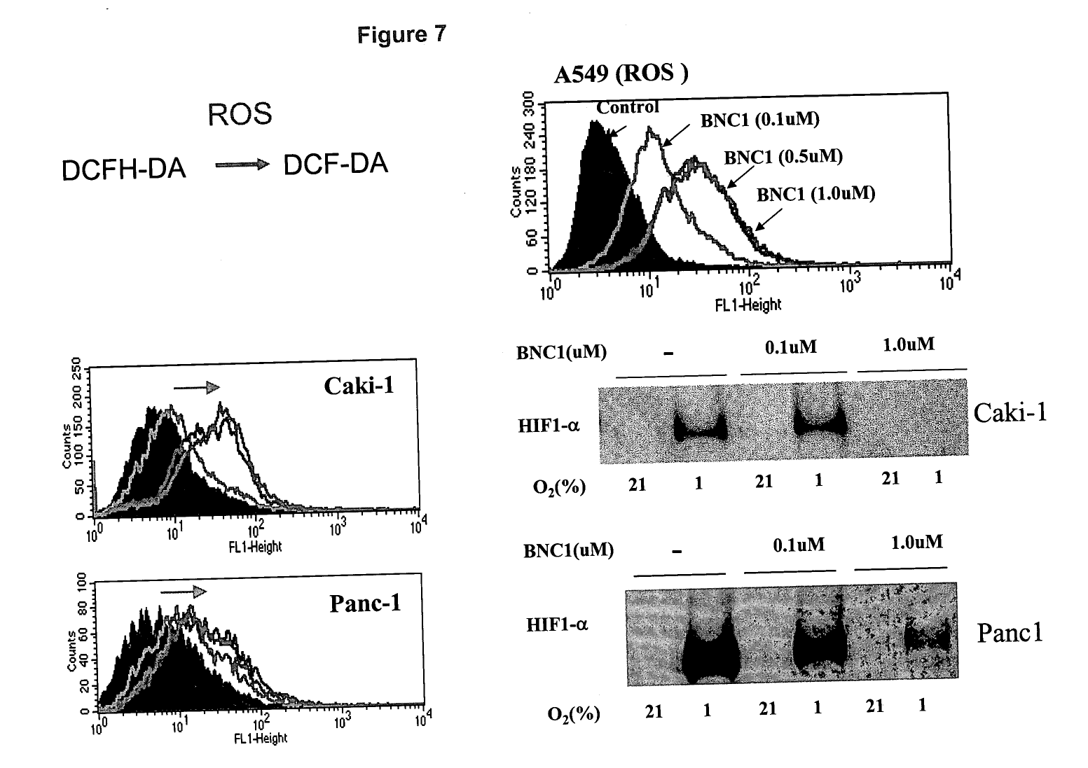

In a related study, tumor cell line A549(ROS) were incubated in normoxia in the absence (control) or presence of different amounts of BNC1 for 4 hrs. Thirty minutes prior to the termination of incubation period, 2,7-dichlorofluorescin diacetate (CFH-DA, 10 mM) was added to the cells and incubated for the last 30 min at 37° C. The ROS levels were determined by FACS analysis. HIF1α protein accumulation in Caki-1 and Panc-1 cells was determined by western blotting after incubating the cells for 4 hrs in normoxia (21% O₂) or hypoxia (1% O₂) in the presence or absence of BNC1. FIG. 7 indicates that BNC1 induces ROS production (at least as evidenced by the A549(ROS) Sentinel Lines), and inhibits HIF1α protein accumulation in the test cells.

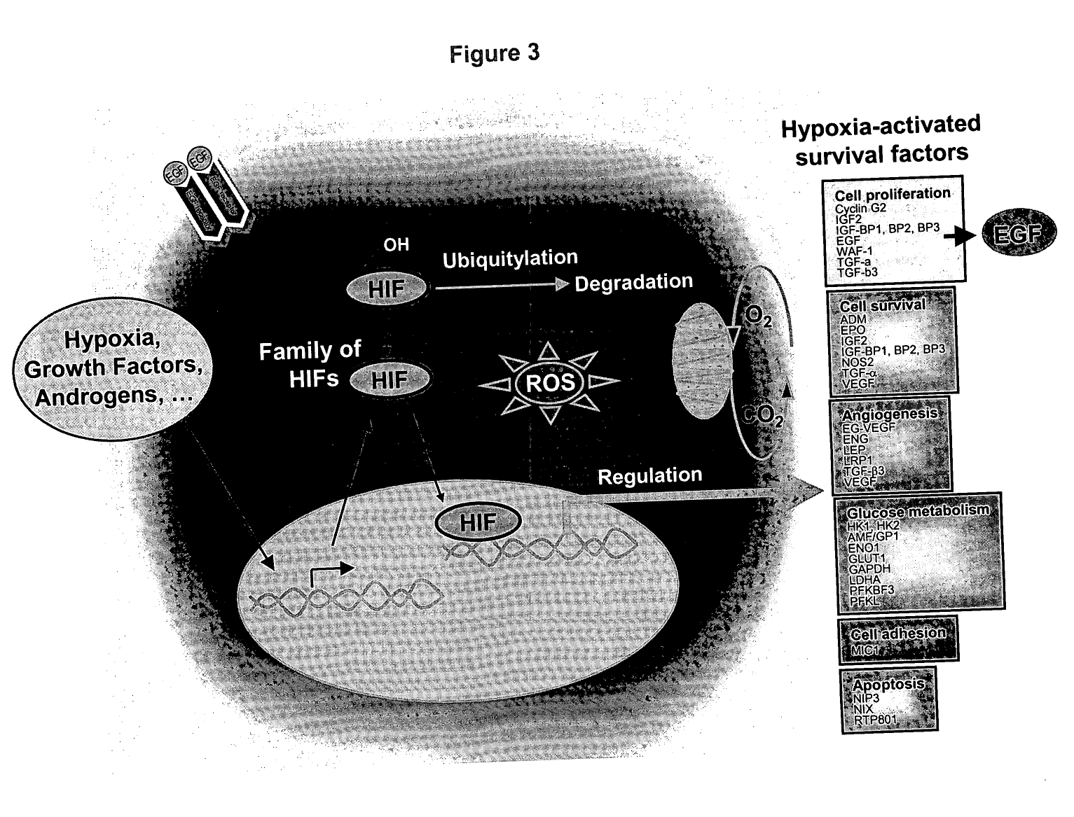

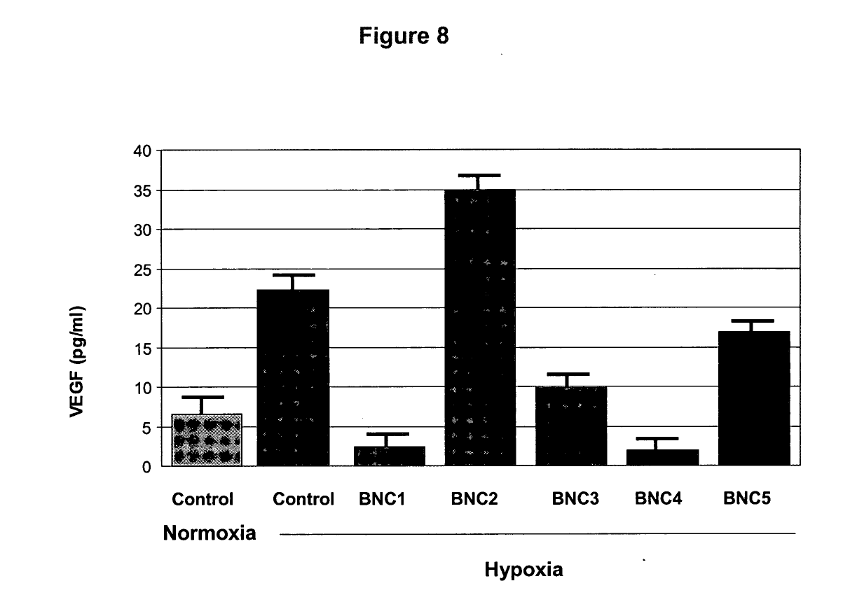

FIG. 8 also demonstrates that the cardiac glycoside compounds BNC1 and BNC4 directly or indirectly inhibits in tumor cells the secretion of the angiogenesis factor VEGF, which is a downstream effector of HIF-1α (see FIG. 3). In contrast, other non-cardiac glycoside compounds, BNC2, BNC3 and BNC5, do not inhibit, and in fact greatly enhances VEGF secretion.

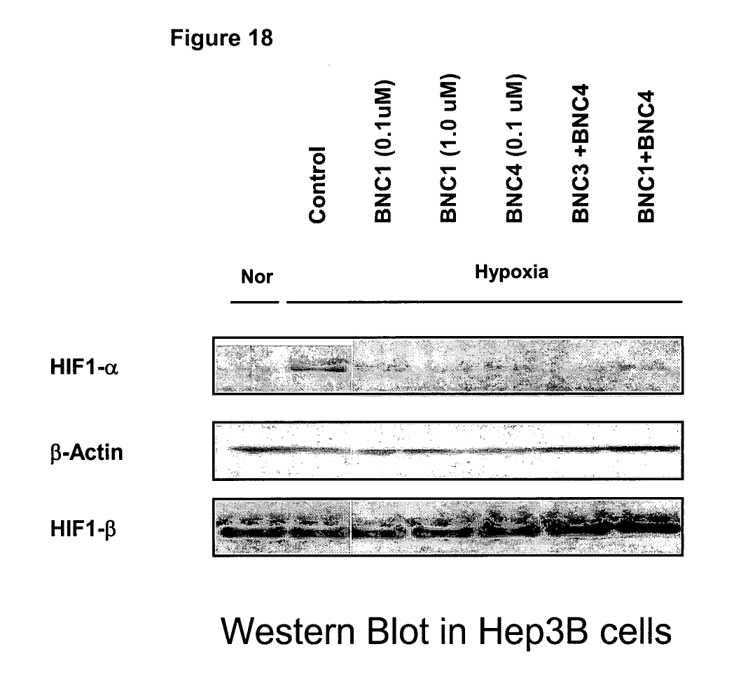

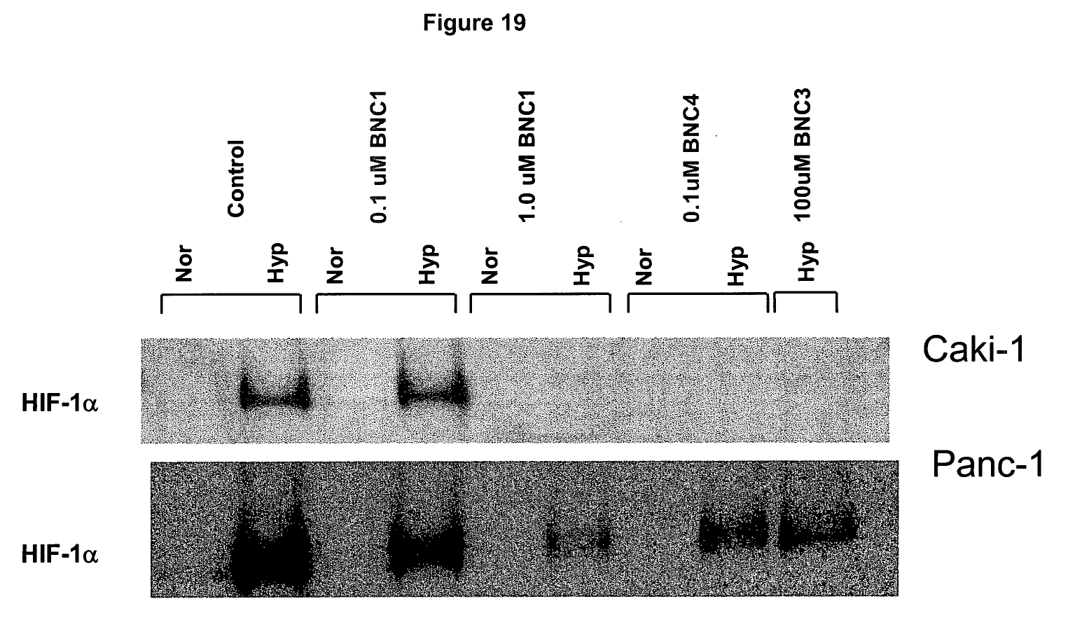

FIGS. 18 and 19 compared the ability of BNC1 and BNC4 in inhibiting hypoxia-mediated HIF1α induction in human tumor cells. The figures show result of immunoblotting for HIF-1α, HIF-1β and β-actin (control) expression, in Hep3B, Caki-1 or Panc-1 cells treated with BNC1 or BNC4 under hypoxia. The results indicate that BNC4 is even more potent (about 10-times more potent) than BNC1 in inhibiting HIF-1α expression.

### Example XI

### Neutralization of Gemcitabine-Induced Stress Response as Measured in A549 Sentinel Line

The cardiac glycoside compounds of the invention were found to be able to neutralize Gemcitabine-induced stress response in tumor cells, as measured in A549 Sentinal Lines.

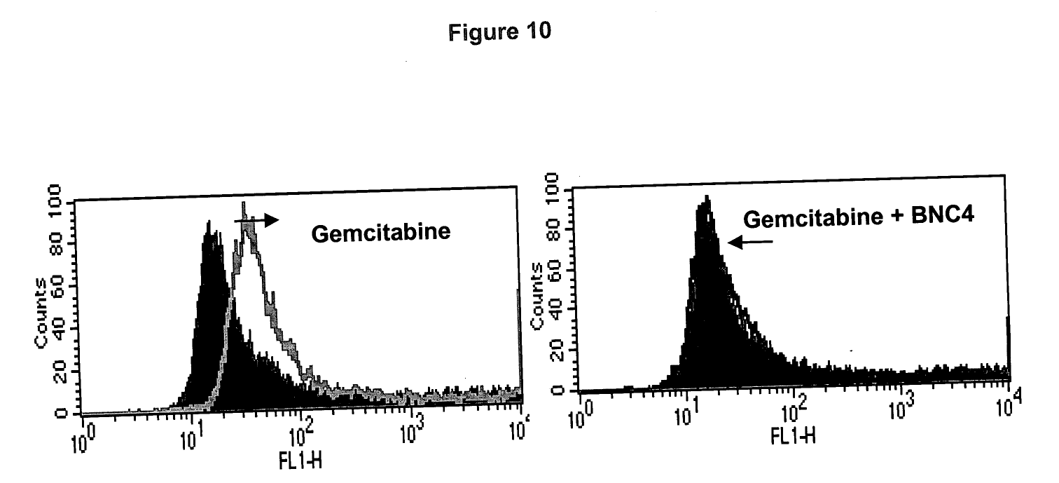

In experiments of FIG. 10, the A549 sentinel line was incubated with Gemcitabine in the presence or absence of indicated Bionaut compounds (including the cardiac glycoside compound BNC4) for 40 hrs. The reporter activity was measured by FACS analysis.

It is evident that at least BNC4 can significantly shift the reporter activity to the left, such that Gemcitabine and BNC4-treated cells had the same reporter activity as that of the control cells. In contrast, cells treated with only Gemcitabine showed elevated reporter activity.

### Example XII

### Effect of BNC1 Alone or in Combination with Standard Chemotherapy on Growth of Xenografted Human Pancreatic Tumors in Nude Mice

To test the ability of BNC1 to inhibit xenographic tumor growth in nude mice, either along or in combination with a standard chemotherapeutic agent, such as Gemcitabine, Panc-1 tumors were injected subcutaneously (sc) into the flanks of male nude mice. After the tumors reached 80 mg in size, osmotic pumps (model 2002, Alzet Inc., flow rate 0.5 μl/hr) containing 20 mg/ml of BNC1 were implanted sc on the opposite sides of the mice. The control animals received pumps containing vehicle (50% DMSO in DMEM). The mice treated with standard chemotherapy agent received intra-peritoneal injections of Gemcitabine at 40 mg/kg every 3 days for 4 treatments (q3d×4). Each data point represent average tumor weight (n=8) and error bars indicate SEM.

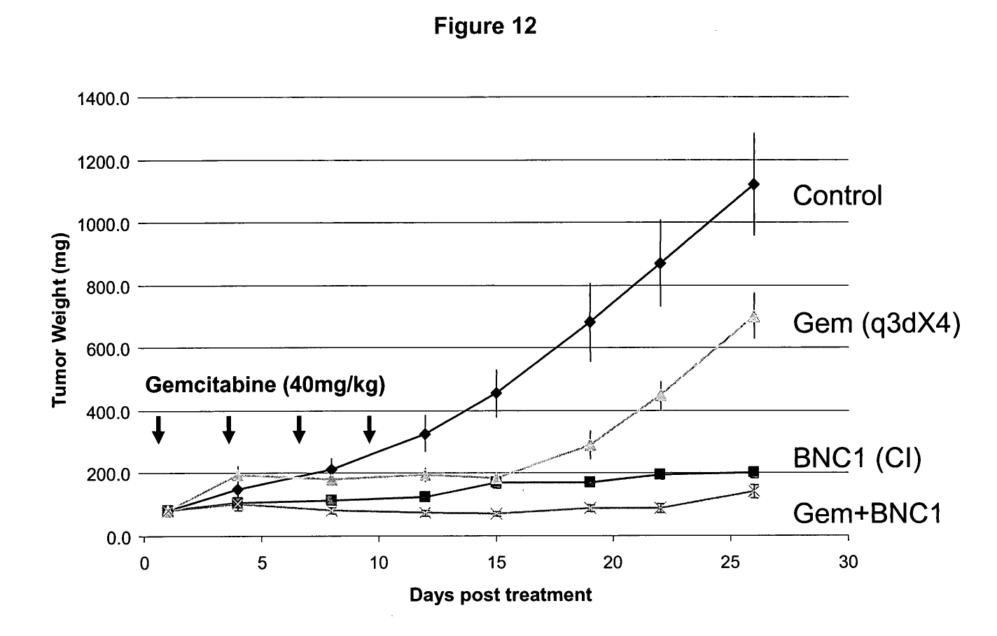

FIG. 12 indicates that, at the dosage tested, BNC1 alone can significantly reduce tumor growth in this model. This anti-tumor activity is additive when BNC1 is co-administered with a standard chemotherapeutic agent Gemcitabine. Results of the experiment is listed below:

| | | Final Tumor | | |
|---|---|---|---|---|
| Group | | weight | | |
| (Animal No.) | Dose/Route | Day 25 (Mean) | SEM | % TGI |
| Control (8) | Vehicle/i.v. | 1120.2 | 161.7 | — |
| BNC1 (8) | 20 mg/ml; | 200 | 17.9 | 82.15 |
| | s.c.; C.I. | | | |
| Gemcitabine | 40 mg/kg; | 701.3 | 72.9 | 37.40 |
| (8) | q3d × 4 | | | |
| BNC1 + | Combine both | 140.8 | 21.1 | 87.43 |
| Gem (8) | | | | |

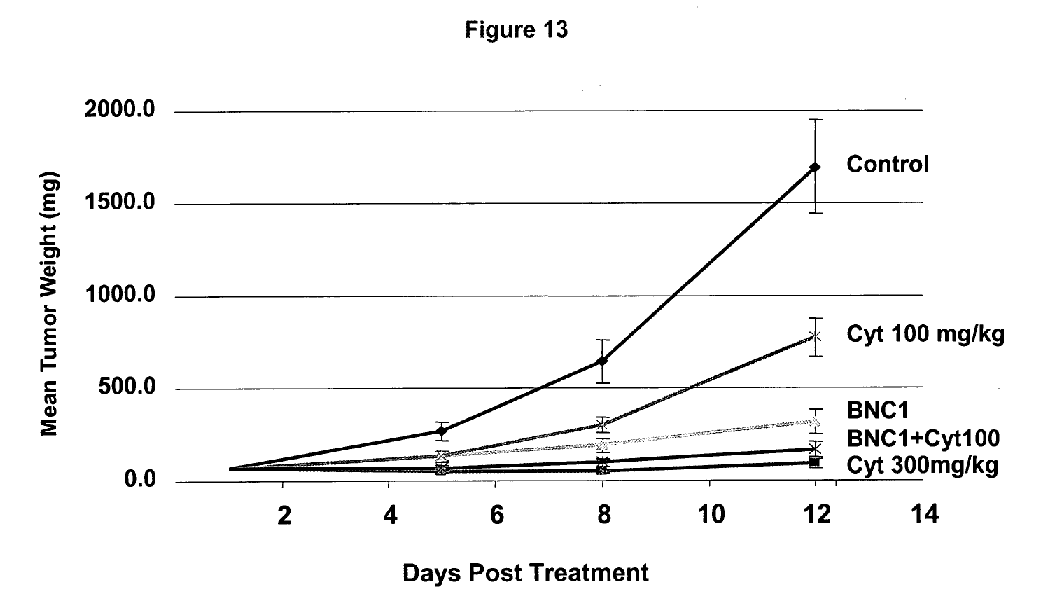

Similarly, in the experiment of FIG. 13, BNC1 (20 mg/ml) was delivered by sc osmotic pumps (model 2002, Alzet Inc.) at 0.5 μl/hr throughout the study. Cytoxan (q1d×1) was injected at 100 mg/kg (Cyt 100) or 300 mg/kg (Cyt 300). The results again shows that BNC1 is a potent anti-tumor agent when used alone, and its effect is additive with other chemotherapeutic agents such as Cytoxan. The result of this study is listed in the table below:

| | | Final Tumor | | |
|---|---|---|---|---|
| Group | | weight | | |
| (Animal No.) | Dose/Route | Day 22 (Mean) | SEM | % TGI |
| Control (10) | Vehicle/i.v. | 1697.6 | 255.8 | — |
| BNC1 (10) | 20 mg/ml; | 314.9 | 67.6 | 81.45 |
| | s.c.; C.I. | | | |
| Cytoxan300 (10) | 300 mg/ml; | 93.7 | 24.2 | 94.48 |
| | ip; qd × 1 | | | |
| Cytoxan100 (10) | 100 mg/ml; | 769 | 103.2 | 54.70 |
| | ip; qd × 2 | | | |
| BNC1 + | Combine both | 167 | 39.2 | 90.16 |
| Cytoxan100 (10) | | | | |

In yet another experiment, the anti-tumor activity of BNC1 alone or in combination with Carboplatin was tested in A549 human non-small-cell-lung carcinoma. In this experiment, BNC1 (20 mg/ml) was delivered by sc osmotic pumps (model 2002, Alzet Inc.) at 0.5 μl/hr throughout the study. Carboplatin (q1d×1) was injected at 100 mg/kg (Carb).

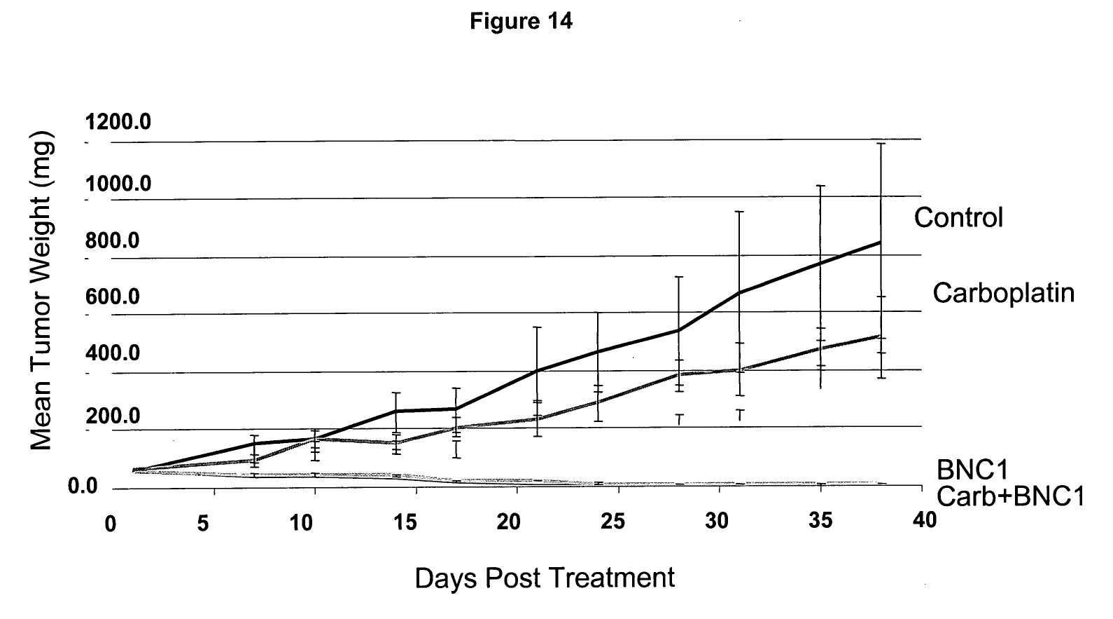

FIG. 14 confirms that either BNC1 alone or in combination with Carboplatin has potent anti-tumor activity in this tumor model. The detailed results of the experiment is listed in the table below:

| | | % Weight | Final Tumor | | |
|---|---|---|---|---|---|
| Group | | Change | weight Day | | |
| (Animal No.) | Dose/Route | at Day 38 | 38 (Mean) | SEM | % TGI |
| Control (8) | Vehicle/i.v. | 21% | 842.6 | 278.1 | — |
| BNC1 (8) | 20 mg/ml; | 21% | 0.0 | 0.0 | 100.00 |
| | s.c.; C.I. | | | | |
| Carboplatin | 100 mg/kg; | 16% | 509.75 | 90.3 | 39.50 |
| (8) | ip; qd × 1 | | | | |
| BNC1 + | Combine | 0% | 0.0 | 0.0 | 100.00 |
| Carb (8) | both | | | | |

Notably, in both the BNC1 and BNC1/Carb treatment group, none of the experimental animals showed any signs of tumor at the end of the experiment, while all 8 experimental animals in control and Carb only treatment groups developed tumors of significant sizes.

Thus the cardiac glycoside compounds of the invention (e.g. BNC1) either alone or in combination with many commonly used chemotherapeutic agents (e.g. Carboplatin, Gem, Cytoxan, etc.) has potent anti-tumor activities in various xenographic animal models of pancreatic cancer, renal cancer, hepatic, and non-small cell lung carcinoma.

### Example XIII

### Determining Minimum Effective Dose

Given the additive effect of the subject cardiac glycosides with the traditional chemotherapeutic agents, it is desirable to explore the minimal effective doses of the subject cardiac glycosides for use in conjoint therapy with the other anti-tumor agents.

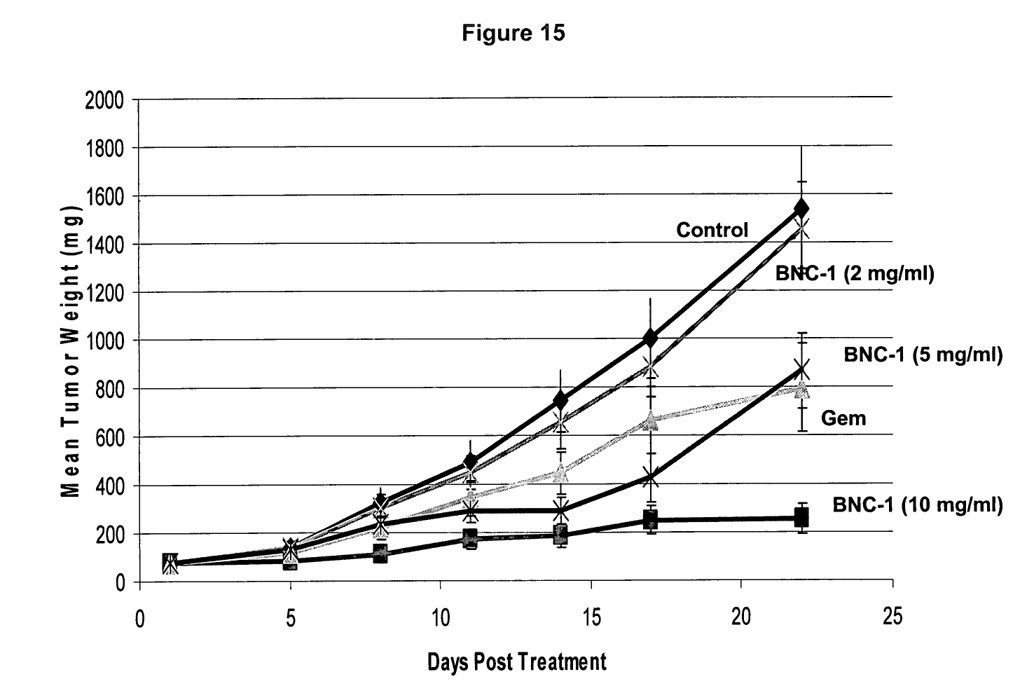

FIG. 15 shows the titration of the exemplary cardiac glycoside BNC1 to determine its minimum effective dose, effective against Panc-1 human pancreatic xenograft in nude mice. BNC1 (sc, osmotic pumps) was first tested at 10, 5 and 2 mg/ml. Gem was also included in the experiment as a comparison.

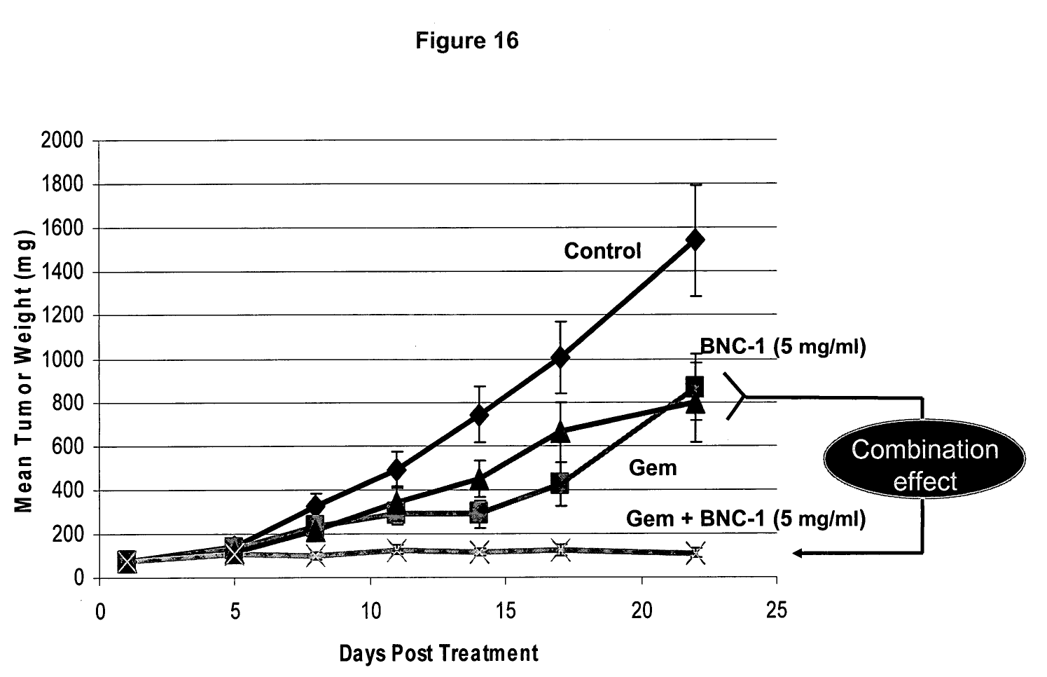

FIG. 16 shows that combination therapy using both Gem and BNC1 produces a combination effect, such that sub-optimal doses of both Gem and BNC1, when used together, produce the maximal effect only achieved by higher doses of individual agents alone.

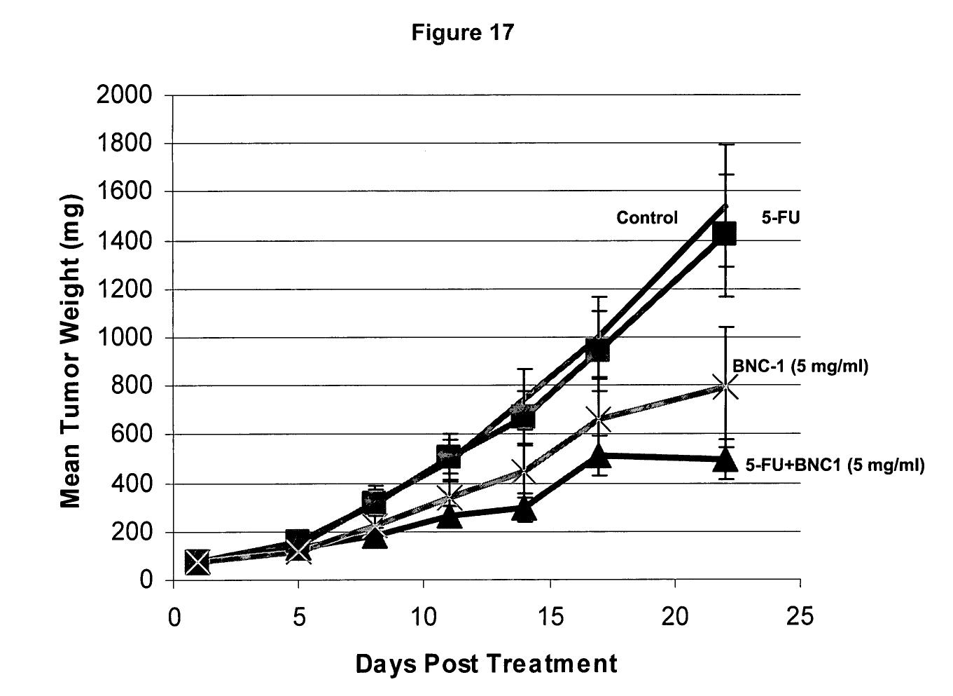

A similar experiment was conducted using BNC1 and 5-FU, and the same combination effect was seen (see FIG. 17).

Similar results are also obtained for other compounds (e.g. BNC2) that are not cardiac glycoside compounds (data not shown).

### Example XIV

### BNC4 Inhibits HIF-1α Induced under Normoxia by PHD Inhibitor

As an attempt to study the mechanism of BNC4 inhibition of HIF-1α, we tested the ability of BNC1 and BNC4 to inhibit HIF-1α expression induced by a PHD inhibitor, L-mimosone (see FIG. 6), under normoxia condition.

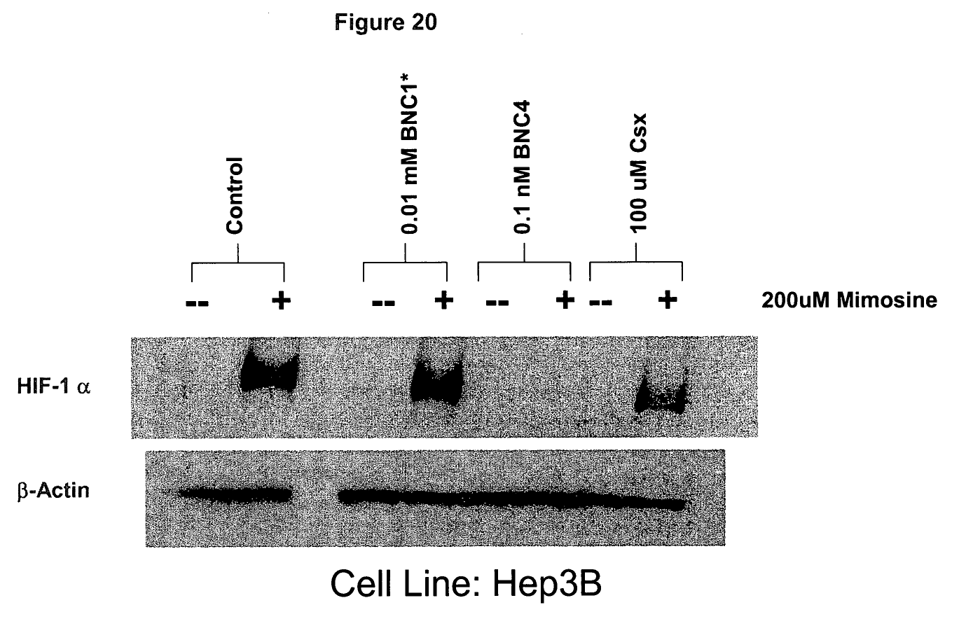

In the experiment represented in FIG. 20, Hep3B cells were grown under normoxia, but were also treated as indicated with 200 μM L-mimosone for 18 h in the presence or absence of BNC1 or BNC4. Abundance of HIF1α and β-actin was determined by western blotting.

The results indicate that L-mimosone induced HIF-1α accumulation under normoxia condition, and addition of BNC4 eliminated HIF-1α accumulation by L-mimosone. At the low concentration tested, BNC1 did not appear to have an effect on HIF-1α accumulation in this experiment. While not wishing to be bound by any particular theory, the fact that BNC4 can inhibit HIF-1α induced under normoxia by PHD inhibitor indicates that the site of action by BNC4 probably lies down stream of prolyl-hydroxylation.

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

1. A pharmaceutical formulation comprising a Na⁺/K⁺-ATPase inhibitor and an anti-cancer agent that induces an hypoxic stress response in tumor cells, formulated in a pharmaceutically acceptable excipient and suitable for use in humans to treat a neoplastic disorder.

2. A kit for treating a patient having a neoplastic disorder, comprising a Na⁺/K⁺-ATPase inhibitor and an anti-cancer agent that induces an hypoxic stress response in tumor cells, each formulated in premeasured doses for conjoint administration to a patient.

3. A method for treating a patient having a neoplastic disorder comprising administering to the patient an effective amount of a Na⁺/K⁺-ATPase inhibitor and an anti-cancer agent that induces an hypoxic stress response in tumor cells.

4. A method for promoting treatment of patients having a neoplastic disorder, comprising packaging, labeling and/or marketing a Na⁺/K⁺-ATPase inhibitor to be used in conjoint therapy for treating a patient having a neoplastic disorder with an anti-cancer agent that induces an hypoxic stress response in tumor cells.

5. A method for promoting treatment of patients having a neoplastic disorder, comprising packaging, labeling and/or marketing an anti-cancer agent that induces an hypoxic stress response in tumor cells to be used in conjoint therapy with a Na⁺/K⁺-ATPase inhibitor for treating a patient having a neoplastic disorder.

6. The pharmaceutical formulation of claim 1, wherein the Na⁺/K⁺-ATPase inhibitor is a cardiac glycoside.

7. The pharmaceutical formulation of claim 6, wherein the cardiac glycoside in combination with the anti-cancer agent has an IC₅₀ and/or an EC₅₀ for killing one or more different cancer cell lines that is at least 2 fold less than the IC₅₀ and/or an EC₅₀, respectively, of the cardiac glycoside alone.

8. The pharmaceutical formulation of claim 6, wherein the cardiac glycoside is represented by the general formula: 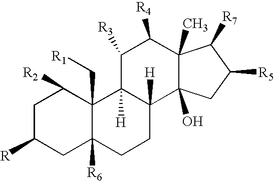
wherein
R represents a glycoside of 1 to 6 sugar residues;
R₁ represents hydrogen, —OH or ═O;
R₂, R₃, R₄, R₅, and R₆ each independently represents hydrogen or —OH; and
R₇ represents 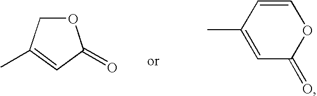
which cardiac glycoside has an IC₅₀ for killing one or more different cancer cell lines of 500 nM or less.

9. The pharmaceutical formulation of claim 6, wherein the cardiac glycoside comprises a steroid core with either a pyrone substituent at C17 (the “bufadienolides form”), or a butyrolactone substituent at C17 (the “cardenolide” form).

10. The pharmaceutical formulation of claim 6, wherein the cardiac glycoside is ouabain or proscillaridin.

11. The pharmaceutical formulation of claim 6, wherein the anti-cancer agent induces redox-sensitive transcription.

12. The pharmaceutical formulation of claim 11, wherein the anti-cancer agent induces HIF-1α-dependent transcription.

13. The pharmaceutical formulation of claim 11, wherein the anti-cancer agent induces expression of one or more of cyclin G2, IGF2, IGF-BP1, IGF-BP2, IGF-BP3, EGF, WAF-1, TGF-α, TGF-β3, ADM, EPO, IGF2, EG-VEGF, VEGF, NOS2, LEP, LRP1, HK1, HK2, AMF/GP1, ENO1, GLUT1, GAPDH, LDHA, PFKBF3, PKFL, MIC1, NIP3, NIX and/or RTP801.

14. The pharmaceutical formulation of claim 6, wherein the anti-cancer agent induces mitochondrial dysfunction and/or caspase activation.

15. The pharmaceutical formulation of claim 6, wherein the anti-cancer agent induces cell cycle arrest at G2/M in the absence of said Cardiac glycoside.

16. The pharmaceutical formulation of claim 6, wherein said anti-cancer agent is an inhibitor of chromatin function.

17. The pharmaceutical formulation of claim 16, wherein said anti-cancer agent is a DNA topoisomerase inhibitor.

18. The pharmaceutical formulation of claim 16, wherein said anti-cancer agent is a microtubule inhibiting drug.

19. The pharmaceutical formulation of claim 6, wherein said anti-cancer agent is a DNA damaging agent.

20. The pharmaceutical formulation of claim 6, wherein said anti-cancer agent is an antimetabolite.

21. The pharmaceutical formulation of claim 6, wherein said antimetabolite is a nucleoside analog that modulates intracellular CTP and/or dCTP metabolism.

22. The pharmaceutical formulation of claim 21, wherein the nucleoside analog is gemcitabine.

23. The pharmaceutical formulation of claim 6, wherein said anti-cancer agent is a DNA synthesis inhibitor.

24. The pharmaceutical formulation of claim 6, wherein said anti-cancer agent is a DNA binding agent.
